SEQUENCE LISTING (9 sequences)

SEQ ID NO: 1 - DNA, 19 bp, Artificial Sequence
  Description of Artificial Sequence Synthetic primer
tcggagtgaacggatttgg

SEQ ID NO: 2 - DNA, 20 bp, Artificial Sequence
  Description of Artificial Sequence Synthetic primer
ccagagttaaaagcagccct

SEQ ID NO: 3 - DNA, 23 bp, Artificial Sequence
  Description of Artificial Sequence Synthetic probe
acgcagtcctctccagtggcgaa

SEQ ID NO: 4 - DNA, 23 bp, Artificial Sequence
  Description of Artificial Sequence Synthetic primer
agaagaagacatcccaccagtca

SEQ ID NO: 5 - DNA, 21 bp, Artificial Sequence
  Description of Artificial Sequence Synthetic primer
agatcacgtcatcgcacaaca

SEQ ID NO: 6 - DNA, 24 bp, Artificial Sequence
  Description of Artificial Sequence Synthetic probe
aacggcctcaggtaccatgaccga

SEQ ID NO: 7 - DNA, 20 bp, Artificial Sequence
  Description of Artificial Sequence Synthetic primer
cccaaccagcgtgacaactt

SEQ ID NO: 8 - DNA, 21 bp, Artificial Sequence
  Description of Artificial Sequence Synthetic primer
ccttctcgtgccagatcatca

SEQ ID NO: 9 - DNA, 23 bp, Artificial Sequence
  Description of Artificial Sequence Synthetic probe
acgcagtcctctccagtggcgaa

(19) United States
(12) Patent Application Publication
(10) Pub. No.: US 2014/0222152 A1
(43) Pub. Date: Aug. 7, 2014

(54) IMPLANTABLE INTERVERTEBRAL DISC DEVICES AND USES THEREOF

(71) Applicant: TUFTS UNIVERSITY, Medford, MA (US)

(72) Inventors: David L. Kaplan, Concord, MA (US); Biman B. Mandal, Kolkata (IN)

(73) Assignee: Tufts University, Medford, MA (US)

(21) Appl. No.: 13/760,826

(22) Filed: Feb. 6, 2013

(51) Int. Cl. A61F 2/44; A61L 27/36

(52) U.S. Cl. CPC A61F 2/442; A61L 27/3604
USPC 623/17.16; 493/394

(57) ABSTRACT

Provided herein are implantable intervertebral disc devices, and methods of making the same. For example, an implantable intervertebral disc device comprises an engineered annulus fibrosus (AF) scaffold that can mimic its native-like cross alignment of pores/channels within the AF region, and can be served as a scaffolding material for cell and matrix alignment. Accordingly, methods of treating a disease or disorder associated with degeneration of an intervertebral disc in a subject are also provided herein, e.g., by replacing the degenerated intervertebral disc of the subject with an implantable intervertebral disc device described herein.

### GOVERNMENT SUPPORT

This invention was made with government support under Grant No. EB002520 awarded by the National Institutes of Health. The government has certain rights in the invention.

### TECHNICAL FIELD OF THE DISCLOSURE

The inventions provided herein generally relate to implantable intervertebral discs and uses thereof, e.g., for treatment of a disease or disorder associated with degeneration of an intervertebral disc.

### BACKGROUND

Low back pain (LBP) is generally associated with degeneration of the intervertebral disc (ND) (Anderson, 1986). IVD degeneration is characterized, in its late stages, by progressive microstructural derangement of the annulus fibrosus (AF) extracellular matrix. Damage to the AF is attributed to mechanical causes, biological remodeling, loss of nutrition, and accumulation of cellular waste products (Iatridis et al., 2005). Current treatment modalities involve conservative management (e.g., mediation and physical therapy) or surgical intervention (e.g., spine fusion, total disc replacement or nucleus pulposus (NP) replacement).

However, the general focus of these methods is to gain symptomatic relief by removal of disc tissue, without specifically identifying the underlying biological problem. In addition, these surgical procedures have limited success rates and they are not applicable to all patients (Hegewald et al., 2008). This issue has led to interest in biological repair of damaged disc tissues, using tissue-engineering methods. ND tissue engineering presents the opportunity to restore the functionality of the ND by repairing or replacing the degenerated tissue (O'Halloran and Pandit, 2007).

For successful cell-based tissue engineering, cells should interact with an appropriate scaffolding material that closely mimics the structural, biological and mechanical functions of native ECM (Venugopal and Ramakrishna, 2005). Therefore, the goal of regenerating a disc tissue should not only be to achieve restoration of anatomical morphology, but also to restore function. Engineering a functional replacement for the AF of the IVD is contingent, in part, upon recapitulation of the AF structure, composition and mechanical properties. While a wide variety of biomaterials have been used in articular cartilage tissue engineering, fewer biomaterials have been used for disc tissue engineering (Frenkel and Di Cesare, 2004; Raghunath et al., 2007). AF tissue-engineering approaches have utilized scaffolds of collagen (Sato et al., 2003), agarose (Gruber et al., 2006), collagen-GAG (Rong et al., 2002), alginate-chitosan (Shao and Hunter, 2007) and polyglycolic acid and polylactic acid (Mizuno et al., 2004). Although these scaffolds were able to support cell growth and desired phenotype, the scaffolds did not recapitulate the architecture of the AF (Chang et al., 2010). Accordingly, there is a strong need to develop an engineered intervertebral disc that can mimic the native structure of an IVD, or particularly the AF region of an IVD.

### SUMMARY

Embodiments described herein are based on, at least in part, engineering a native-like annulus fibrosus (AF) region of an intervertebral disc (IVD) with a silk-based matrix. For example, a silk-based matrix can be configured to mimic lamellar and cross-alignment features of an AF region of an IVD. The inventors have demonstrated, in a specific embodiment, that a silk-based toroidal disc scaffold that possesses lamellar and cross-alignment features similar to the AF region of a native IVD can be produced by arranging laminar silk-based scaffold strips (e.g., silk-based scaffold strips formed by a directional freezing method) around a vertical axis in alternating directions to create cross alignment of lamellar channels in the resulting silk-based toroidal disc scaffold, wherein the laminar silk-based scaffold strips comprise aligned (lamellar) channels or pores at a pre-determined angle (e.g., at an angle of about 30° with respect to the transverse plane of the toroidal disc scaffold). The inventors have further demonstrated that the lamellar silk-based scaffolds support cell seeding and proliferation (including cell penetration), as well as production of extracellular matrix (e.g., but not limited to, collagen and GAG) within the scaffold, to form an AF-like tissue. Thus, the inventions provided herein relate to implantable intervertebral disc devices, methods of making the same, and methods of using the same, e.g., for treatment of a disease or disorder associated with degeneration of an intervertebral disc.

In one aspect, described herein relates to an implantable intervertebral disc device comprising a silk-based toroidal disc scaffold with lamellar porous structures. In one embodiment, the implantable intervertebral disc device comprises a silk-based toroidal disc scaffold, wherein the silk-based toroidal disc scaffold comprises on its circumferential surface at least two concentric layers of laminar silk scaffold strips, e.g., a first lamellar silk scaffold strip and a second lamellar silk scaffold strip.

Laminar silk scaffold strips are generally elongated pieces of silk-based matrices comprising laminar porous structures. However, the laminar silk scaffold strips are not silk-based meshes or fabrics (e.g., silk-based fibers knitted in a certain pattern). In some embodiments, the laminar silk scaffold strips can be rectangular in shape. In these embodiments, the laminar silk scaffold strips can have any dimension. For example, in some embodiments, the laminar silk scaffold strips can each have a width or height adjusted to match with the height of an intervertebral disc to be repaired or replaced.

As each circumference surface of the silk-based toroidal disc scaffold generally increases in length with its radius, the total length of the laminar silk scaffold strip in each subsequent layer can increase with increasing radius of the circumferential surface. In some embodiments, the first laminar silk scaffold strip and/or the second laminar silk scaffold strip can each independently be a single continuous piece having a dimension in sufficient length to form a circumferential surface at a different radius of the silk-based toroidal disc scaffold. In other embodiments, the first laminar silk scaffold strip and/or the second laminar silk scaffold strip forming a distinct circumferential surface at a different radius of the silk-based toroidal disc scaffold can each be independently formed by connecting together at least two or more (e.g., 2, 3, 4 or more) shorter laminar silk scaffold strips having substantially the same or similar alignment of its porous structures such that the combined length is sufficient to form a circumferential surface of the silk-based toroidal disc scaffold.

The thickness of the laminar silk scaffold strips used to form a silk-based toroidal disc scaffold can be varied, e.g., depending on the thickness of lamellar structures of a native annulus fibrosus to be replaced. For example, the laminar silk scaffold strips can each independently have a thickness ranging from about 50 μm to about 2 mm, or from about 100 μm to about 1 mm, or from about 250 μm to about 750 μm. In general, a silk-based toroidal disc scaffold having a certain size would comprise more concentric layers of the laminar silk scaffold strips when thinner, rather than thicker, laminar silk scaffold strips are used.

In accordance with embodiments of various aspects described herein, the silk-based toroidal disc scaffold comprises a first laminar silk scaffold strip forming a first concentric circumferential surface layer, and a second laminar silk scaffold strip forming a second concentric circumferential surface layer. In some embodiments, the first laminar silk scaffold strip comprises at least about 5% of its first porous structures substantially aligned to an alignment axis (e.g., an alignment axis forming a predetermined angle with a reference axis such as the bottom edge of the first laminar scaffold strip or with a reference plane such as a transverse plane of the resulting silk-based toroidal disc scaffold. In some embodiments, the first laminar silk scaffold strip can comprise at least about 10%, at least about 20%, at least about 30%, at least about 40%, at least about 50%, at least about 75%, at least about 90% or more, of the first porous structures substantially aligned to an alignment axis (e.g., an alignment axis forming a predetermined angle with a reference axis such as the bottom edge of the first laminar scaffold strip or with a reference plane such as a transverse plane of the resulting silk-based toroidal disc scaffold. In some embodiments, the predetermined angle formed between the alignment axis and the reference axis or the reference plane can range from about 10° to about 80°, about 15° to about 60°, about 20° to about 40°, or about 25° to about 35°. In one embodiment, the predetermined angle formed between the alignment axis and the reference axis or the reference plane can be about 25° to about 35° (e.g., about 30°).

In various embodiments, the second laminar silk scaffold strip is wrapped circumferentially around the first laminar silk scaffold strip in a manner such that at least about 5% of second porous structures present in the second laminar silk scaffold strip are substantially aligned at an angle of about 20° to about 160° (including, e.g., about 40° to about 150°, about 60° to about 145°, about 80° to about 140°, about 100° to about 140°, or about 110° to about 130°) with respect to the aligned first porous structures. In some embodiments, at least about 10%, at least about 20%, at least about 30%, at least about 40%, at least about 50%, at least about 75%, at least about 90% or more, of the second porous structures can be substantially aligned at the angle of about 20° to about 160° (including, e.g., about 40° to about 150°, about 60° to about 145°, about 80° to about 140°, about 100° to about 140°, and about 110° to about 130°) with respect to the aligned first porous structures. Stated another way, in some embodiments, at least about 5%, including, e.g., at least about 10%, at least about 20%, at least about 30%, at least about 40%, at least about 50%, at least about 75%, at least about 90% or higher, of the porous structures between any two successive concentric layers (e.g., the first and the second concentric layers) can be substantially oriented at an angle of about 20° to about 160° with each other, e.g., at an angle of about 40° to about 150°, about 60° to about 145°, about 80° to about 140°, about 100° to about 140°, or about 110° to about 130° with respect to each other. In one embodiment, at least about 5%, including, e.g., at least about 10%, at least about 20%, at least about 30%, at least about 40%, at least about 50%, at least about 75%, at least about 90% or higher, of the porous structures between any two successive concentric layers (e.g., the first and the second concentric layers) can be substantially oriented at an angle of about 120°.

The porous structures of the silk-based toroidal disc scaffold can have a pore size of any dimension, and/or a cross-section of any shape. In some embodiments, the porous structures can have a pore size of about 10 μm to about 500 μm, or about 100 μm to about 250 μm. In some embodiments, the cross-sectional shape of a porous structure can include, but are not limited to, a square, a rectangle, a circle, an oval, a polygon, an irregular shape, or any combinations thereof.

The spacing between any two successive lamellar silk-based scaffold strips (designated as “inter-lamellar spacing” herein) can vary with a number of factors, e.g., but not limited to pore size of the porous structures present in a laminar silk-based scaffold strip. In some embodiments, the inter-lamellar spacing can range from about 10 μm to about 500 μm, or from about 150 μm to about 250 μm.

The silk-based toroidal disc scaffold can be configured to have any size, e.g., depending on the size of a native annulus fibrosus to be replaced. The size of the silk-based toroidal disc scaffold can be varied with a number of factors, including, e.g., but not limited to, the number and/or thickness of the concentric laminar silk scaffold strip layers, the pore size of the porous structures and the inter-lamellar spacing. In some embodiments, the silk-based toroidal disc scaffold can comprise on its circumferential surface any number of concentric layers of the lamellar silk scaffold strips, e.g., any number greater than 2, greater than 3, greater than 4, greater than 5, or more. In general, keeping other factors constant, the greater the number of the concentric layers used to form a silk-based toroidal disc scaffold, the larger the size of the resulting silk-based toroidal disc scaffold. In some embodiments, the silk-based toroidal disc scaffold can comprise a range of about 2 to about 30 concentric layers, or about 10 to about 20 concentric layers. In some embodiments, the silk-based toroidal disc scaffold can comprise a number of concentric layers sufficient to yield a desirable thickness of the silk-based toroidal disc scaffold (e.g., comparable to the thickness of an annulus fibrosus structure of a subject to be replaced).

A native intervertebral disc generally comprises an outer annulus fibrosus, which surrounds the inner gel-like nucleus pulposus. Accordingly, in some embodiments, an intervertebral disc device can further comprise a biocompatible gel surrounded by the silk-based toroidal disc scaffold. The biocompatible gel can comprise any biopolymer or biocompatible polymer. In some embodiments, the biocompatible gel can comprise silk fibroin.

In some embodiments, the implantable intervertebral disc device can further comprise an active agent. For example, the silk-based toroidal disc scaffold and/or the biocompatible gel can each independently comprise an active agent, e.g., to facilitate repair and/or regeneration of the IVD, to minimize implant rejection, and/or to enhance cell growth and/or cell production of extracellular matrix. Examples of an active agent can include, but are not limited to, a cell, a therapeutic agent, an anesthetic, a cell growth factor, a peptide, a peptidomimetic, an antibody or a portion thereof, an antibody-like molecule, nucleic acid (e.g., but not limited to, DNA, RNA, siRNA, shRNA), a polysaccharide, an aptamer, an enzyme, a receptor antagonist or agonist, a hormone, an autogenous bone marrow, an antibiotic, an antimicrobial agent, a small molecule, a cell attachment agent, a macrophage-skewing agent or any combinations thereof. Non-limiting examples of a cell attachment agent can include hyaluronic acid, collagen, crosslinked hyaluronic acid/collagen, an integrin-binding molecule, chitosan, elastin, fibronectin, vitronectin, laminin, proteoglycans, any derivatives thereof, any peptide or oligosaccharide variants thereof, and any combinations thereof. In one embodiment, the active agent present in the implantable intervertebral disc device can comprise at least one cell (e.g., an intervertebral disc-associated cell). The cell(s) can be present in the silk-based toroidal disc scaffold, the biocompatible gel, or both.

While implantable intervertebral disc devices described herein can be produced by any known methods in the art, in some embodiments, the implantable intervertebral disc devices can be produced by one or more embodiments of the production method described below. Accordingly, another aspect provided herein relates to methods of producing an implantable intervertebral disc device. In one embodiment, the method of producing an implantable intervertebral disc device described herein comprises: (a) providing a first laminar silk scaffold strip and a second laminar silk scaffold strip, wherein the first and the second laminar silk scaffold strips each comprises at least about 5% of its porous structures substantially aligned at a predetermined angle; and (b) layering the first laminar silk scaffold strip and the second laminar silk scaffold strip to form a laminate composite, wherein the second laminar silk scaffold strip is positioned such that the aligned porous structures of the second laminar silk scaffold strip are substantially oriented at an angle of about 20° to about 160° (including, e.g., about 40° to about 150°, about 60° to about 145°, about 80° to about 140°, about 100° to about 140°, or about 110° to about 130°, or an angle of about 120°) with respect to the aligned porous structures of the first laminar silk scaffold strip; wherein the laminate composite forms a circumferential surface of a toroidal disc scaffold.

While in some embodiments, the first laminar silk scaffold strip and the second laminar silk scaffold strip can form a laminate composite prior to forming a circumferential surface of a toroidal disc scaffold, the first laminar silk scaffold strip and the second laminar silk scaffold strip can, in alternative embodiments, sequentially wrap around the same vertical axis to form a circumferential surface of a toroidal disc scaffold while forming a laminate composite. Accordingly, in alternative embodiments, the method of producing an implantable intervertebral disc device can comprise: (a) providing a first laminar silk scaffold strip and a second laminar silk scaffold strip, wherein the first and the second laminar silk scaffold strips each comprises at least about 5% of its porous structures substantially aligned at a predetermined angle; (b) wrapping with a first laminar silk scaffold strip circumferentially around a vertical axis; (c) wrapping with a second laminar silk scaffold strip circumferentially around the first laminar silk scaffold strip in a manner such that the aligned porous structures of the second laminar silk scaffold strip are substantially oriented at an angle of about 20° to about 160° (including, e.g., about 40° to about 150°, about 60° to about 145°, about 80° to about 140°, about 100° to about 140°, or about 110° to about 130°, or an angle of about 120°) with respect to the aligned porous structures of the first laminar silk scaffold strip, thereby producing an implantable intervertebral disc device comprising a silk-based toroidal disc scaffold formed from the first and the second laminar silk scaffold strips.

In some embodiments, the laminate composite or the first laminar silk scaffold strip can be circumferentially wrapped around a substantially circular disc element, which provides the vertical axis. In some embodiments, the substantially circular disc element can comprise a biocompatible gel or hydrogel. For example, a biocompatible gel or hydrogel can comprise silk fibroin.

In some embodiments, the method can further comprise seeding at least one cell into at least a portion of an intervertebral disc device described herein. In some embodiments, the method can further comprise culturing at least one cell seeded into at least a portion of an intervertebral disc device described herein.

Laminar silk scaffold strips described herein can be produced by any methods known in the art. In one embodiment, a laminar silk scaffold strip is produced by a method comprising: exposing a silk fibroin solution to unidirectional freezing; and lyophilizing the frozen silk fibroin solution, thereby forming a laminar silk scaffold. In some embodiments, the method of producing a laminar silk scaffold strip can further comprise reducing the laminar silk scaffold (e.g. if the scaffold dimension is larger than a dimension of an annulus fibrosus) into strips of smaller dimensions, e.g., the first laminar silk scaffold strip and the second laminar silk scaffold strip.

In some embodiments, the silk fibroin solution for production of a laminar silk scaffold strip can further comprise a water-soluble pore-forming agent, e.g., but not limited to, sodium alginate. In such embodiments, the method of producing a laminar silk scaffold strip can further comprise removing the water-soluble pore-forming agent from the resultant laminar silk scaffold.

The solubility and/or degradation of silk in an aqueous solution can be generally reduced by inducing beta-sheet crystallinity in the silk. Accordingly, in some embodiments where decreased solubility and/or degradation is desirable, the laminar silk scaffold or the laminar silk scaffold strips can be subjected to a post-treatment for increasing beta sheet content in silk fibroin, e.g., but not limited to, water annealing.

A further aspect provided herein relates to uses of the implantable intervertebral disc devices described herein. For example, one or more embodiments of the implantable intervertebral disc devices described herein can be used for treatment of a degenerated intervertebral disc in a subject. In one embodiment, provided herein is a method of treating a disease or disorder associated with degeneration of an intervertebral disc in a subject, wherein the method comprises replacing the degenerated intervertebral disc of the subject with one or more embodiments of the implantable intervertebral disc device described herein.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIGS. 1A-1B are schematic diagrams showing exemplary methods for silk scaffold fabrication. FIG. 1A illustrates an exemplary method of fabricating lamellar scaffolds. FIG. 1B illustrates an exemplary method of fabricating porous scaffolds.

FIGS. 2A-2B are sets of images showing structural features of one or more embodiments of silk-based scaffolds described herein. FIG. 2A is a set of images showing structural features of an exemplary lamellar silk scaffold. FIG. 2B is a set of images showing structural features of an exemplary porous silk scaffold. Scale bar=200 μm.

FIG. 3 shows FTIR spectra of one or more embodiments of silk-based scaffolds described herein. In the figure, line (a) corresponds to a freeze-dried scaffold; line (b) corresponds to a freeze-dried scaffold with EDAC/NHS; line (c) corresponds to a freeze-dried scaffold, water annealed (lamellar structure); and line (d) corresponds to a freeze-dried scaffold with EDAC/NHS, water annealed (porous structure).

FIGS. 4A-4F are SEM images and live cell staining of porcine AF cells in one or more embodiments of lamellar and porous silk-based scaffolds. FIG. 4A is a SEM image showing one embodiment of a lamellar silk-based scaffold after 1 week of culture. FIG. 4B is a SEM image showing one embodiment of a lamellar silk-based scaffold after 2 weeks of culture. FIG. 4C is a fluorescent image showing live/dead staining of seeded AF cells in one embodiment of a lamellar silk-based scaffold. FIG. 4D is a SEM image showing a porous silk-based scaffold after 1 week of culture. FIG. 4E is a SEM image showing a porous silk-based scaffold after 2 weeks of culture. FIG. 4F is a fluorescent image showing live/dead staining of seeded AF cells in a porous silk-based scaffold. Arrows in the figures indicate proliferating cells. Scale bars=300 μm.

FIGS. 5A-5D are histological images of AF cells after 2 weeks of culture in various embodiments of silk-based scaffolds. FIG. 5A is a hematoxylin and eosin (H&E) stain image of AF cells after 2 weeks of culture in one embodiment of a lamellar silk-based scaffold described herein. FIG. 5B is a hematoxylin and eosin (H&E) stain image of AF cells after 2 weeks of culture in a porous silk-based scaffold. FIG. 5C is an immunohistochemical image for type I collagen produced by AF cells cultured in one embodiment of a lamellar silk-based scaffold described herein. FIG. 5D is an immunohistochemical image for type I collagen produced by AF cells cultured in a porous silk-based scaffold described herein. Dashed arrows indicate cells. Extracellular matrix stained positive (brown) for type I collagen (solid arrow). Scale bars=100 μm.

FIG. 6A-6C are bar graphs showing chemical analysis results of different molecules in silk-based scaffolds. FIG. 6A is a bar graph showing DNA content per a scaffold, for a lamellar and a porous silk-based scaffold, respectively. FIG. 6B is a bar graph showing GAG content per a scaffold, for a lamellar and a porous silk-based scaffold, respectively. FIG. 6C is a bar graph showing total collagen content per a scaffold, for a lamellar and a porous silk-based scaffold, respectively. Data shown as mean±SD from four samples (* p<0.05, ** p<0.01 and *** p<0.001).

FIGS. 7A-7B are bar graphs showing transcript levels of AF tissue differentiation markers in AF cells cultured in various embodiments of silk-based scaffolds. FIG. 7A is a bar graph showing a transcript expression level of colIα1 marker in AF cells cultured in a lamellar or a porous silk-based scaffold. FIG. 7B is a bar graph showing a transcript expression level of aggrecan marker in AF cells cultured in a lamellar or a porous silk-based scaffold. Data quantified by real-time PCR and normalized to GAPDH within the linear range of amplification. Data shown as mean±SD from n=4; *p<0.05, ** p<0.01 and ***p<0.001.

FIGS. 8A-8C are bar graphs showing analysis results of mechanical strength of a lamellar or a porous silk-based scaffold after it has been seeded with AF cells and cultured for an indicated period of time. FIG. 8A is a bar graph showing data of linear elastic modulus between a lamellar or a porous silk-based scaffold after it has been seeded with AF cells and cultured for a day or 2 weeks. FIG. 8B is a bar graph showing data of ultimate tensile strength between a lamellar or a porous silk-based scaffold after it has been seeded with AF cells and cultured for a day or 2 weeks. FIG. 8C is a bar graph showing data of elongation to failure between a lamellar or a porous silk-based scaffold after it has been seeded with AF cells and cultured for a day or 2 weeks.

FIG. 9 is an image showing an exemplary fabricated native-like annulus fibrous (AF) region of an intervertebral disc using a plurality of strips of silk aligned scaffolds described herein.

FIG. 10 is a photograph showing an exemplary custom PDMS device used to fabricate a lamellar or aligned silk-based scaffold.

FIG. 11 is a schematic representation showing an exemplary method to fabricate a lamellar or aligned silk-based scaffold with a custom PDMS device as shown in FIG. 10. A unidirectional freezing method is employed in the PDMS chamber to achieve directional or lamellar channel formation in a silk-based scaffold for intervertebral disc engineering.

FIGS. 12A-12B are images showing exemplary structural features of an aligned/lamellar silk-based scaffold fabricated using a directional freezing method. FIG. 12A is a SEM image showing pore alignment and porosity of channels/pores formed in the aligned/lamellar silk-based scaffold. FIG. 12B is a fluorescent image showing alignment of fluorescent protein-tagged fibroblast cells grown a laminar silk-based scaffold.

FIG. 13 is a set of images showing an exemplary process of fabricating a native-like AF region of an intervertebral disc, e.g., as shown in FIG. 9. The left panel of FIG. 13 is a schematic representation of preparing silk-based scaffold strips with channels/pores aligned at a certain angle to mimic cross alignment at an angle of about 30 degrees with respect to a circumferential direction, as observed in a native-like AF tissue. The right panel of FIG. 13 is a set of images of aligned silk-based scaffolds and cut strips showing orientation of channels/pores aligned at an alternating angle of about 30 degrees with respect to an axis along the length of the strip.

FIG. 14 is a set of images showing different perspectives of a native-like AF region of an engineered intervertebral disc as shown in FIG. 9. The top left panel of FIG. 14 shows exemplary strips of aligned silk-based scaffolds with the orientation of channels/pores aligned at an alternating angle of about 30 degrees with respect an axis along the length of the strips. The right panels of FIG. 14 are images showing an exemplary full-sized annulus fibrosus (AF) region of an intervertebral disc fabricated using the method as shown in FIG. 13, where the AF region, in one embodiment, comprises about 9 layers of aligned silk-based scaffold strips (top right panel) showing a cross aligned structure (e.g., the alignment of channels/pores in each layer is oriented at an alternating angle of about 30 degrees with respect to a circumferential direction, as shown in the middle right and bottom right panels of the figure). The center panel of FIG. 14 is a diagrammatic figure showing alignment of channels/pores in each lamellar silk-based scaffold layer of an engineered AF scaffold. The bottom panel of FIG. 14 is a diagrammatic view showing a portion of two laminar silk scaffold strips oriented in a manner such that the alignment axis of the first laminar silk scaffold strip forms an angle α (or a complementary angle of 180°-α) with the alignment axis of the second laminar silk scaffold strip in order to mimic the cross alignment feature of the AF structure.

FIG. 15 is a set of images showing microtome sections of an exemplary engineered AF scaffold. The microtome sections show 30-degree cross aligned structures within the engineered AF scaffold.

### DETAILED DESCRIPTION OF THE INVENTION

Degeneration of intervertebral discs (IVDs) represents a significant muscular skeletal disease. While scaffolds of synthetic, natural and hybrid biomaterials have been explored as options to restore functionality of the IVD by repairing or replacing the degenerated tissues, the existing scaffolds do not generally recapitulate the architecture (e.g., structure, composition, and/or mechanical properties) of the annulus fibrosis (AF) and thus do not provide optimum or functional AF tissue restoration. Accordingly, there is still a need for an improved implantable intervertebral disc scaffold for functional replacement of a degenerated IVD. To this end, the inventors have engineered, in one embodiment, a silk-based toroidal scaffold that mimics lamellar structures and cross alignment of the lamellae present in an AF tissue.

Accordingly, embodiments described herein are based on, at least in part, engineering a native-like annulus fibrosus (AF) region of an intervertebral disc (IVD) with a silk-based matrix. For example, a silk-based matrix can be configured to mimic lamellar and cross-alignment features of an AF region of an IVD. The inventors have demonstrated, in a specific embodiment, that a silk-based toroidal disc scaffold that possesses lamellar and cross-alignment features similar to the AF region of a native IVD can be produced by arranging laminar silk-based scaffold strips (e.g., silk-based scaffold strips formed by a directional freezing method) around a vertical axis in alternating directions to create cross alignment of lamellar channels in the resulting silk-based toroidal disc scaffold, wherein the laminar silk-based scaffold strips comprise aligned (lamellar) channels or pores at a pre-determined angle (e.g., at an angle of about 30° with respect to the transverse plane of the toroidal disc scaffold). The inventors have further demonstrated that the lamellar silk-based scaffolds support cell seeding and proliferation (including cell penetration), as well as production of extracellular matrix (e.g., but not limited to, collagen and GAG) within the scaffold, to form an AF-like tissue. Thus, the inventions provided herein relate to implantable intervertebral disc devices, methods of making the same, and methods of using the same, e.g., for treatment of a disease or disorder associated with degeneration of an intervertebral disc.

### Exemplary Embodiments of Implantable Intervertebral Disc Devices

In one aspect, described herein relates to an implantable intervertebral disc device comprising a silk-based toroidal (or annulus) disc scaffold with lamellar porous structures. In one embodiment, the implantable intervertebral disc device comprises a silk-based toroidal (or annulus) disc scaffold, wherein the silk-based toroidal disc scaffold comprises on its circumferential surface at least two layers of laminar silk scaffold strips, e.g., a first lamellar silk scaffold strip and a second lamellar silk scaffold strip. In some embodiments, said at least two layers of the laminar silk scaffold strips are concentric to each other and form the circumferential surface of the toroidal disc scaffold.

As used herein, the term “scaffold” generally refers to a structure or framework made of a biocompatible material. For example, the scaffold can be configured to adopt a structure or framework of a toroidal (or annulus) disc, e.g., a circular disk with a hollow center. In one embodiment, the scaffold can be configured to adopt a structure or a framework of a strip, which is termed as “a scaffold strip” herein. In one embodiment, a scaffold refers to a three-dimensional biocompatible structure that mimics at least a functional unit of an intervertebral disc (e.g., annulus fibrosus, and/or nucleus pulposus) In some embodiments, a scaffold (e.g., a lamellar scaffold) can provide support or anchor sites to any cells seeded therein over a period of time, e.g., at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 1 year or longer. In some embodiments, a scaffold (e.g., a lamellar scaffold) can support cell proliferation and/or facilitate a biological process (e.g., production of extracellular matrix) of cells seeded therein over a period of time, e.g., at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 1 year or longer. In some embodiments, a scaffold (e.g., a lamellar scaffold) can guide alignment or orientation of extracellular matrix (e.g., common extracellular matrix present in an annulus fibrous such as collagen) produced or deposited by cells (e.g., annulus fibrosus-associated cells), which are attached to the scaffold in alignment with lamellar pore orientation.

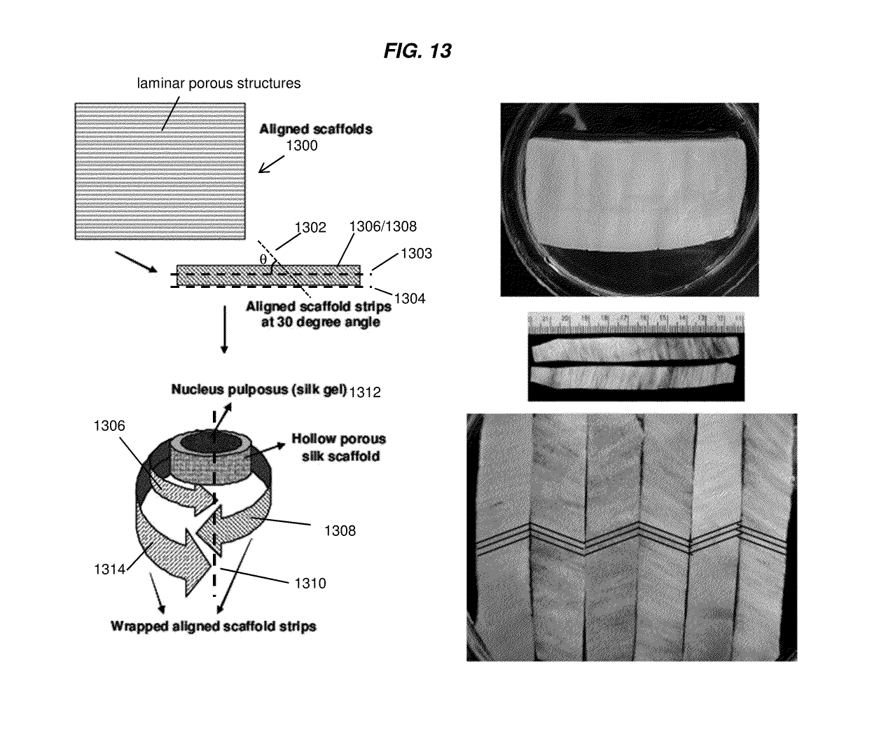

As used herein, the term “lamellar” with respect to structural characterization of a feature in a scaffold generally refers to arrangement, orientation or organization of pores or channels in a scaffold. For example, in some embodiments, a lamellar silk scaffold can refer to a silk-based scaffold comprising a lamellar porous structure. The term “lamellar porous structure” as used herein refers to pores and/or channels that are substantially aligned to an alignment axis. As used herein, the term “alignment axis” refers to the alignment direction of the pores and/or channels. By way of example only, as shown in FIG. 13, the alignment axis 1302 of a laminar silk scaffold strip can be defined by orientation of the pores and/or channels at a pre-determined angle θ to a reference axis such as a transverse axis 1303 of the laminar silk scaffold strip or the bottom edge of the laminar scaffold strip 1304. In some embodiments, the alignment axis 1302 of a laminar silk scaffold strip can be defined by orientation of the pores and/or channels at a pre-determined angle θ to a reference plane, e.g., a transverse plane of the resulting silk-based toroidal disc scaffold. The pre-determined angle θ between the alignment axis 1302 and the reference axis (e.g., 1303, 1304) or the reference plane can range from about 10° to about 80°, about 15° to about 60°, about 20° to about 40°, or about 25° to about 35°. In some embodiments, the pre-determined angle θ between the alignment axis 1302 and the reference axis (e.g., 1303, 1304) or the reference plane can range from about 20° to about 40°. In some embodiments, the pre-determined angle θ between the alignment axis 1302 and the reference axis (e.g., 1303, 1304) or the reference plane can range from about 25° to about 35°. In one embodiment, the pre-determined angle θ between the alignment axis 1302 and the reference axis or the reference plane (e.g., 1303, 1304) is about 30°.

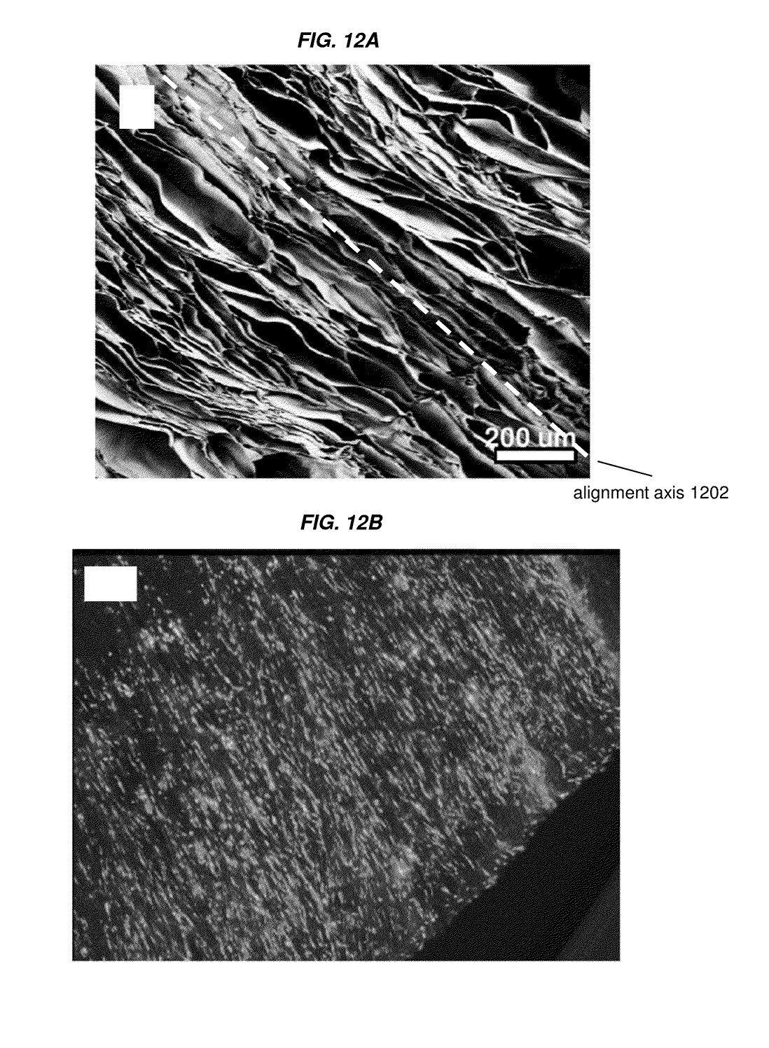

Stated another way, a lamellar silk scaffold can be a multi-layered scaffold comprising sheets (or lamellae) of a silk-based scaffolding material, where the spacing between the lamellae determines the pore or channel size. FIG. 12A shows a SEM image of pore alignment within an exemplary lamellar silk scaffold.

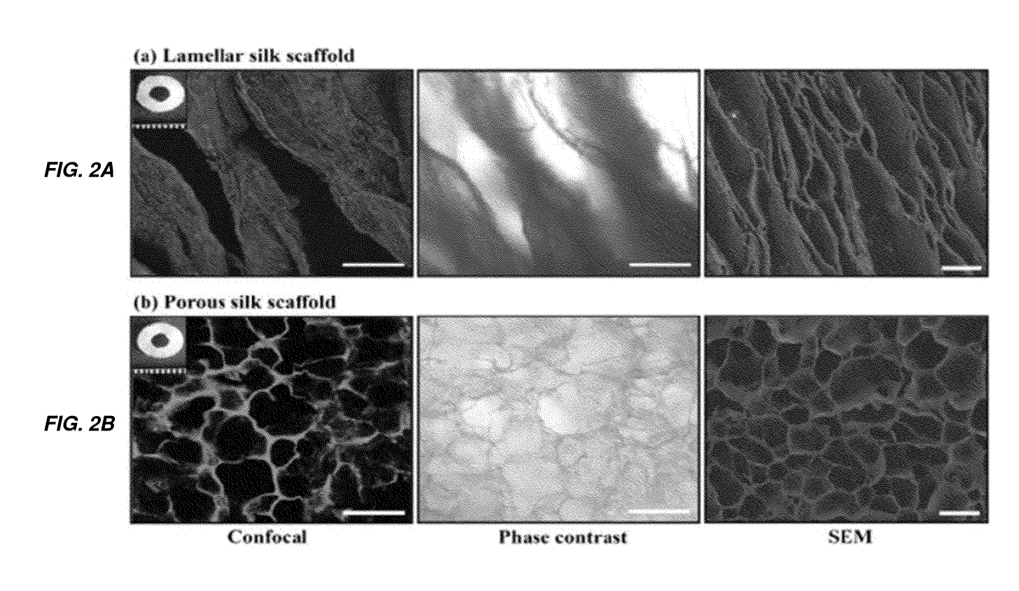

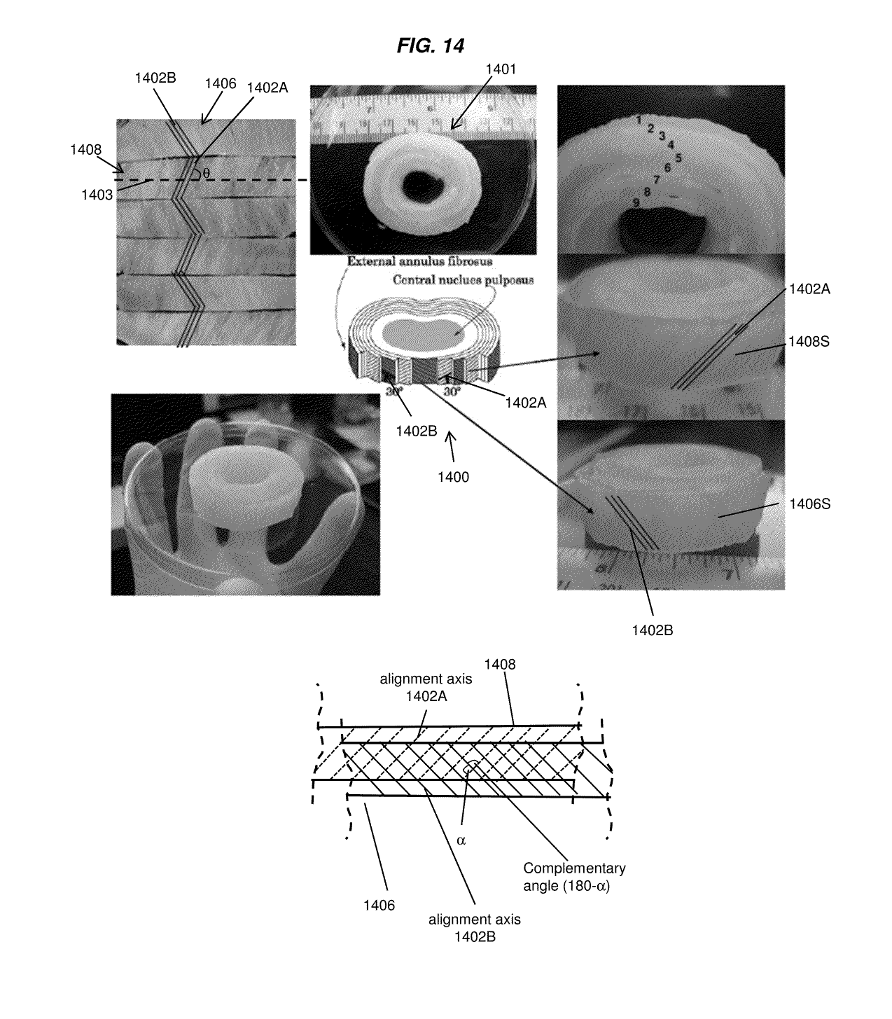

As used herein, the term “substantially aligned” refers to a general directional orientation of the porous structures (e.g., pores and/or channels) present in a scaffold, without necessary regard to the degree of alignment. That is, the term refers to the porous structures generally running in the same direction, or tending to run in the same average direction. It is used to connote anisotropy of the pore and/or channel orientation and to distinguish from a random orientation. FIGS. 2A and 12A show the porous structures that are substantially aligned, running in the same average direction, despite variations in the degree of orientation of the individual porous structures, while FIG. 2B shows random pore orientation. However, it is not necessary that individual porous structures that are substantially aligned run parallel within a layer. To be substantially aligned requires a measurable degree of directionality. One non-limiting way to quantify this degree of alignment is to describe the deviation of individual porous structures (e.g., pores and/or channels) from an alignment axis, e.g., 1202 in FIG. 12A, 1302 in FIG. 13, or 1402A and 1402B in FIG. 14. For examples, the porous structures (e.g., pores and/or channels) are substantially aligned when the porous structures are within 60° of the alignment axis, including, within 50°, within 40°, within 30°, within 20°, within 10°, or within 5° of the alignment axis.

As used herein, the phrase “laminar silk scaffold strip” describes a silk-based scaffold in a form of strip (e.g., a rectangular strip) comprising a laminar porous structure described herein. In accordance with various embodiments described herein, these laminar silk scaffold strips, at least in part, form a circumference surface of the silk-based toroidal disc scaffold described herein. However, the laminar silk scaffold strips do not comprise silk-based meshes, fabrics, or cloths (e.g., silk-based fibers knitted in a certain pattern).

The laminar silk scaffold strips can have any dimension, for example, depending on the size of a silk-based toroidal disc scaffold to be constructed and/or the size of a degenerated intervertebral disc to be repaired or replaced. By way of example only, in some embodiments, the laminar silk scaffold strips can each have a width or height adjusted to match with the height of an intervertebral disc to be repaired or replaced.

As each circumference surface of the silk-based toroidal disc scaffold generally increases in perimeter with its radius, the total length of the laminar silk scaffold strip in each subsequent outer layer can increase with increasing radius of the circumferential surface. In some embodiments, the first laminar silk scaffold strip and/or the second laminar silk scaffold strip can each independently be a single continuous piece having a dimension in sufficient length to form a circumferential surface at a specified radius of the silk-based toroidal disc scaffold. In other embodiments, the first laminar silk scaffold strip and/or the second laminar silk scaffold strip forming a distinct circumferential surface at a specified radius of the silk-based toroidal disc scaffold can each independently comprise at least two or more (e.g., 2, 3, 4 or more) shorter laminar silk scaffold strips having substantially the same or similar alignment of its porous structures such that the combined length is sufficient to form a circumferential surface of the silk-based toroidal disc scaffold.

The thickness of the laminar silk scaffold strips used to form a silk-based toroidal disc scaffold can be varied, e.g., depending on the thickness of lamellar structures of a native annulus fibrosus to be replaced, and/or dimensions of the lamellar porous structures. For example, the laminar silk scaffold strips can each independently have a thickness ranging from about 50 μm to about 2 mm, or from about 100 μm to about 1 mm, or from about 250 μm to about 750 μm. In general, a silk-based toroidal disc scaffold having a certain size would comprise more concentric layers of the laminar silk scaffold strips when thinner, rather than thicker, laminar silk scaffold strips are used.

In accordance with embodiments of various aspects described herein, the silk-based toroidal disc scaffold comprises at least two laminar silk scaffold strips, overlapping so as to be oriented one over another to form the circumference surface of the toroidal disc scaffold. Other embodiments provide for more than two laminar silk scaffold strips, e.g., in a multi-layer construct. By way of example only, as shown in FIGS. 13 and 14, the silk-based toroidal disc scaffold comprises a first laminar silk scaffold strip 1306, 1406 forming a first concentric circumferential surface layer 1306S, 1406S, and a second laminar silk scaffold strip 1308, 1408 forming a second concentric circumferential surface layer 1308S, 1408S.

In various embodiments, at least two laminar silk scaffold strips are arranged such that the alignment axis 1402B of the first laminar scaffold strip 1406 forms an angle α with the alignment axis 1402A of the second laminar scaffold strip 1408. The angle α defined by the alignment axes 1402A, 1402B on any consecutive laminar scaffold strips (e.g., 1406, 1408) can be in the range from about 20° to about 160°, or from about 30° to about 140°, or from about 35° to about 120°, or from about 40° to about 100°, or from about 40° to about 80°, or from about 50° to about 70°. In some embodiments, the angle α defined by the alignment axes 1402A, 1402B on any consecutive laminar scaffold strips (e.g., 1406, 1408) can be in the range from about 50° to about 70°. In one embodiment, the angle α defined by the alignment axes 1402A, 1402B on any consecutive laminar scaffold strips (e.g., 1406, 1408) is in the range of about 60°.

Stated another way, in various embodiments, at least two laminar silk scaffold strips can be arranged such that the alignment axis 1402B of the first laminar scaffold strip 1406 forms a complementary angle (180°-α) with the alignment axis 1402A of the second laminar silk scaffold strip 1408. For example, the second laminar silk scaffold strip can be wrapped circumferentially around the first laminar silk scaffold strip in a manner such that at least some laminar porous structures present in the second laminar silk scaffold strip are substantially aligned at a complementary angle (180°-α) of about 20° to about 160° (including, e.g., about 40° to about 150°, about 60° to about 145°, about 80° to about 140°, about 100° to about 140°, or about 110° to about 130°) with respect to the first laminar porous structures. In some embodiments, at least about 5% of second porous structures present in the second laminar silk scaffold strip can be substantially aligned at the complementary angle (180°-α) of about 20° to about 160° (including, e.g., about 40° to about 150°, about 60° to about 145°, about 80° to about 140°, about 100° to about 140°, or about 110° to about 130°) with respect to the first laminar porous structures. In some embodiments, at least about 10%, at least about 20%, at least about 30%, at least about 40%, at least about 50%, at least about 75%, at least about 90% or more, of the second porous structures can be substantially aligned at the complementary angle (180°-α) of about 20° to about 160° (including, e.g., about 40° to about 150°, about 60° to about 145°, about 80° to about 140°, about 100° to about 140°, or about 110° to about 130°) with respect to the first laminar porous structures. In one embodiment, at least about 5%, at least about 10%, at least about 20%, at least about 30%, at least about 40%, at least about 50%, at least about 75%, at least about 90% or more, of the second porous structures can be substantially aligned at the complementary angle (180°-α) of about 120° with respect to the first laminar porous structures. Stated another way, in some embodiments, at least about 5%, including, e.g., at least about 10%, at least about 20%, at least about 30%, at least about 40%, at least about 50%, at least about 75%, at least about 90% or higher, of the porous structures between any two successive concentric layers (e.g., the first and the second concentric layers) can be substantially oriented at the complementary angle (180°-α) angle of about 20° to about 160° with each other, e.g., at an angle of about 40° to about 150°, about 60° to about 145°, about 80° to about 140°, about 100° to about 140°, or about 110° to about 130° with respect to each other. In one embodiment, at least about 5%, including, e.g., at least about 10%, at least about 20%, at least about 30%, at least about 40%, at least about 50%, at least about 75%, at least about 90% or higher, of the porous structures between any two successive concentric layers (e.g., the first and the second concentric layers) can be substantially oriented at an angle of about 120°.

In some embodiments, the angle α or the complementary angle (180°-α) defined by the alignment axes on any two consecutive laminar scaffold strips can vary across the silk-based toroidal disc scaffold described herein. As an non-limiting example, a first pair of laminar scaffold strips can be oriented such that the alignment axes of these scaffold strips are oppositely oriented at an angle θ of about 20° to about 40° (e.g., about 30°) with respect to a reference axis (e.g., a transverse axis), whereas a second pair laminar scaffold strips, within the same multiplayer construct, can be oriented oppositely at a different angle θ (e.g., at about 45°) with respect to a reference axis (e.g., a transverse axis).

The first laminar silk scaffold strip (e.g., 1306, 1406) can comprise a laminar porous structure, e.g., pores and/or channels that are substantially aligned to an alignment axis. In some embodiments, the first laminar silk scaffold strip can comprise at least about 5% of its first porous structures (e.g., pores and/or channels) substantially aligned to its alignment axis described herein (e.g., an alignment axis 1302 forming a pre-determined angle θ with a reference axis such as a transverse axis 1303 of the laminar silk scaffold strip or the bottom edge of the laminar scaffold strip 1304, or with a reference plane such as a transverse plane of the resulting silk-based toroidal disc scaffold). In some embodiments, the first laminar silk scaffold strip can comprise at least about 10%, at least about 20%, at least about 30%, at least about 40%, at least about 50%, at least about 75%, at least about 90% or more, of the first porous structures substantially aligned to its alignment axis (e.g., an alignment axis 1302 forming a pre-determined angle θ with a reference axis such as a transverse axis 1303 of the laminar silk scaffold strip or the bottom edge of the laminar scaffold strip 1304, or with a reference plane such as a transverse plane of the resulting silk-based toroidal disc scaffold).

The second laminar silk scaffold strip (e.g., 1308, 1408) can comprise a laminar porous structure, e.g., pores and/or channels that are substantially aligned to an alignment axis. In some embodiments, the second laminar silk scaffold strip can comprise at least about 5% of its second porous structures (e.g., pores and/or channels) substantially aligned to its alignment axis described herein (e.g., an alignment axis 1402A forming a pre-determined angle θ with a reference axis such as a transverse axis 1403 of the laminar silk scaffold strip or the bottom edge of the laminar scaffold strip 1408, or with a reference plane such as a transverse plane of the resulting silk-based toroidal disc scaffold). In some embodiments, the second laminar silk scaffold strip can comprise at least about 10%, at least about 20%, at least about 30%, at least about 40%, at least about 50%, at least about 75%, at least about 90% or more, of the second porous structures substantially aligned to its alignment axis (e.g., an alignment axis 1402A forming a pre-determined angle θ with a reference axis such as a transverse axis 1403 of the laminar silk scaffold strip or the bottom edge of the laminar scaffold strip 1408, or with a reference plane such as a transverse plane of the resulting silk-based toroidal disc scaffold).

In some embodiments, the first and/or the second laminar silk scaffold strip can each independently comprise more than one distinct laminar porous structure defined by at least two distinct alignment axes. By way of example only, the first and/or the second laminar silk scaffold strip can each independently comprise at least two distinct laminar porous structures defined by their respective alignment axes of different orientations or directions (e.g., the alignment axes of these distinct laminar porous structures can be oppositely oriented, e.g., at about 30° with respect to a reference axis such as a transverse axis of the first or the second laminar silk scaffold strip).

In some embodiments, the silk-based toroidal disc scaffold can further comprise at least one layer comprising random pores (e.g., pores without any preferred alignment orientation). In these embodiments, the silk-based toroidal disc scaffold can comprise at least one layer of a non-lamellar silk scaffold strip containing random pores. In some embodiments, random pores can be present in one or more laminar silk scaffold strips. For example, in some embodiments, a laminar silk scaffold strip can comprise at least one pore that is not substantially aligned to the alignment axis of the laminar silk scaffold strip.

In some embodiments, the silk-based toroidal disc scaffold or the laminar silk scaffold strip described herein can be configured to have any porosity, depending on the desired properties and/or morphology of native tissue structures. For example, in some embodiments, the silk-based toroidal disc scaffold or the laminar silk scaffold strip described herein can have a porosity of at least about 1%, at least about 3%, at least about 5%, at least about 10%, at least about 15%, at least about 20%, at least about 25%, at least about 30%, at least about 40%, at least about 50%, at least about 60%, at least about 70%, at least about 80%, at least about 90% or higher. In some embodiments, the porosity can range from about 70% to about 99%, or from about 80% to about 98%. As used herein, the term “porosity” is generally used to describe a structure having a network of pores, channels or void spaces (which can, for example, be openings, interstitial spaces or other channels) throughout its volume. The term “porosity” is a measure of void spaces in a material, and is a fraction of volume of voids over the total volume, as a percentage between 0 and 100% (or between 0 and 1). The pore size and total porosity values can be quantified using conventional methods and models known to those of skill in the art. For example, the pore size and porosity can be measured by standardized techniques, such as mercury porosimetry and nitrogen adsorption. One of ordinary skill in the art can determine the optimal porosity within the silk-based toroidal disc scaffold or the laminar silk scaffold strip for various purposes. For example, in some embodiments the porosity and/or pore size of the silk-based toroidal disc scaffold or the laminar silk scaffold strip described herein can be optimized based on the desired degradation rate or volume retention rate of the silk fibroin-based matrices, release profiles of an active agent, if any, from the silk fibroin-based matrices, and/or the structural morphology of the tissue to be repaired or augmented.

The porous structures of the silk-based toroidal disc scaffold can have a pore size of any dimension, and/or a cross-section of any shape. In some embodiments, the porous structures can have a pore size of about 1 μm to about 1000 μm, or about 10 μm to about 500 μm, or about 100 μm to about 250 μm. In one embodiment, the porous structures can have a pore size of about 100 μm to about 250 μm. In some embodiments, the porous structures can have a pore size sufficient to permit infiltration of at least one cell or more, e.g., at least one annulus fibrosus (AF) cell or a plurality of AF cells. The term “pore size” as used herein refers to a dimension of a pore or an average dimension of a plurality of pores. In some embodiments, the pore size can refer to the longest dimension of a pore, e.g., a diameter of a pore having a circular cross section, or the length of the longest cross-sectional chord that can be constructed across a pore having a non-circular cross-section. In other embodiments, the pore size can refer the shortest dimension of a pore. In some embodiments, the cross-sectional shape of a porous structure can include, but are not limited to, a square, a rectangle, a circle, an oval, a polygon, an irregular shape, or any combinations thereof.

The spacing between any two successive lamellar silk-based scaffold strips (termed as “inter-lamellar spacing” herein) can vary with a number of factors, e.g., but not limited to pore size of the porous structures present in a laminar silk-based scaffold strip. In some embodiments, the inter-lamellar spacing can range from about 1 μm to about 1000 μm, or about 10 μm to about 500 μm, or from about 150 μm to about 250 μm. In one embodiment, the inter-lamellar spacing can range from about 150 μm to about 250 μm.

The silk-based toroidal disc scaffold can be configured to have any size, depending on, e.g., the size of a native annulus fibrosus to be replaced, the number and/or thickness of the concentric laminar silk scaffold strip layers, the pore size of the porous structures and/or the inter-lamellar spacing. In some embodiments, the silk-based toroidal disc scaffold can comprise on its circumferential surface any number of concentric layers of the lamellar silk scaffold strips, e.g., any number greater than 2, greater than 3, greater than 4, greater than 5, or more. In general, keeping other factors constant, the greater the number of the concentric layers used to form a silk-based toroidal disc scaffold, the larger the size of the resulting silk-based toroidal disc scaffold. In some embodiments, the silk-based toroidal disc scaffold can comprise a range of about 2 to about 30 concentric layers, or about 10 to about 20 concentric layers. In some embodiments, the silk-based toroidal disc scaffold can comprise a number of concentric layers sufficient to yield a desirable thickness of the silk-based toroidal disc scaffold (e.g., comparable to the thickness of an annulus fibrosus structure of a subject to be replaced).

The intervertebral disc (IVD) is a complex structure that can be separated macroscopically into at least two anatomical zones: the nucleus pulposus (NP), representing a centrally located gelatinous homogeneous mass, and the annulus fibrosus (AF), consisting of concentrically organized layers of collagen fibrils which surround the NP (Kluba et al., 2005). Both tissues contain an abundant matrix of negatively charged proteoglycans entangled with collagen fibers. In particular, the AF consists of an extracellular matrix (ECM) composed of both type I and type II collagen orientated in a lamellar structure with a predominance of type I collagen (Richardson et al., 2006; Wan et al., 2008). The fundamental tension-bearing elements are bundles of type I collagen fibrils, which are arranged obliquely to the axial plane of the disc in discontinuous, approximately concentric lamellae around the nucleus (Marchand and Ahmed, 1990). Proteoglycans, principally aggrecan, represent the second greatest constituent of the disc in terms of dry weight after collagen, constituting 5-8% of the outer annulus and 11-20% of the inner annulus (Feng et al., 2006).

In embodiments of various aspects described herein, the silk-based toroidal disc scaffolds described herein comprise lamellar porous structures oppositely aligned in alternating layers in order to mimic the cross-alignment feature of the annulus fibrosus (AF) structure. Accordingly, unlike some of the existing AF scaffolds, such as alginate/chitosan scaffold (Shao and Hunter (2007)) and biphasic scaffolds (Wan et al. (2008)), the silk-based toroidal disc scaffolds described herein do not comprise fibers, e.g., silk-based fibers, which were used in the existing AF scaffolds to create the cross-alignment and lamellar feature.

A native intervertebral disc generally comprises an outer annulus fibrosus, which surrounds the inner gel-like nucleus pulposus. Accordingly, in some embodiments, an intervertebral disc device described herein can further comprise a biocompatible gel surrounded by the silk-based toroidal disc scaffold. The biocompatible gel can comprise any biopolymer or biocompatible polymer. In some embodiments, the biocompatible gel can comprise silk fibroin. In these embodiments, an intervertebral disc device described herein can further comprise a silk-based gel surrounded by one or more embodiments of the silk-based toroidal disc scaffold. The silk-based gel can be porous or non-porous.

As used herein, the phrase “silk-based” or “silk” in reference to a matrix structure, e.g., a silk-based toroidal disc scaffold, a silk scaffold strip, or a silk-based gel generally refers to a matrix (e.g., a silk-based toroidal disc scaffold, a silk scaffold strip, or a silk-based gel) in which silk (or silk fibroin) constitutes at least about 10% (w/w) of the total matrix, including at least about 20% (w/w), at least about 30% (w/w), at least about 40% (w/w), at least about 50%(w/w), at least about 60%(w/w), at least about 70% (w/w), at least about 80% (w/w), at least about 90% (w/w), at least about 95% (w/w), up to and including 100% (w/w) or any percentages between about 10% (w/w) and about 100% (w/w), of the total matrix. In certain embodiments, the silk matrix (e.g., a silk-based toroidal disc scaffold, a silk scaffold strip, or a silk-based gel) can be substantially formed from silk or silk fibroin. In various embodiments, the silk matrix (e.g., a silk-based toroidal disc scaffold, a silk scaffold strip, or a silk-based gel) can be substantially formed from silk or silk fibroin comprising at least one additive (e.g., an active agent and/or a biocompatible polymer).

In some embodiments, the silk-based matrices described herein (e.g., a silk-based toroidal disc scaffold, a silk scaffold strip, or a silk-based gel) can comprise at least one or more biocompatible and/or biodegradable polymers to form mixed polymer matrices comprising silk or silk fibroin. In some embodiments, one or more biocompatible and/or biodegradable polymers (e.g., two or more biocompatible polymers) can be added to the silk solution prior to forming the silk-based matrices described herein. The biocompatible polymer that can be used herein include, but are not limited to, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, hyaluronic acid and other biocompatible and/or biodegradable polymers. See, e.g., International Application Nos.: WO 04/062697; WO 05/012606, the contents of which are incorporated herein by reference.

In some embodiments, the implantable intervertebral disc device can further comprise an active agent. For example, the silk-based toroidal disc scaffold and/or the biocompatible gel can each independently comprise an active agent, e.g., to facilitate repair and/or regeneration of the IVD, to minimize implant rejection, and/or to enhance cell growth and/or cell production of extracellular matrix. Examples of an active agent can include, but are not limited to, a cell, a therapeutic agent, an anesthetic, a cell growth factor, a peptide, a peptidomimetic, an antibody or a portion thereof, an antibody-like molecule, nucleic acid (e.g., but not limited to, DNA, RNA, siRNA, shRNA), a polysaccharide, an aptamer, an enzyme, a receptor antagonist or agonist, a hormone, an autogenous bone marrow, an antibiotic, an antimicrobial agent, a small molecule, a cell attachment agent, a macrophage-skewing agent or any combinations thereof. Non-limiting examples of a cell attachment agent can include hyaluronic acid, collagen, crosslinked hyaluronic acid/collagen, an integrin-binding molecule, chitosan, elastin, fibronectin, vitronectin, laminin, proteoglycans, any derivatives thereof, any peptide or oligosaccharide variants thereof, and any combinations thereof. Any macrophage-skewing agent that can be used to control inflammation and/or facilitate regeneration of a damaged tissue, e.g., immune cell-modulating agents described in the International Application No. PCT/US12/72275 filed Dec. 31, 2012 entitled “Functionalization of biomaterials to control regeneration and inflammation responses” can be included in the implantable intervertebral disc device described herein.

The active agent can be present in any amount in the implantable intervertebral disc device described herein. In some embodiments, the active agent can be present in the order of micrograms to milligrams to grams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %.

In one embodiment, the active agent present in the implantable intervertebral disc device can comprise at least one or more cells (e.g., stem cells and/or intervertebral disc-associated cells such as an annulus fibrosus (AF) cells). The cell(s) can be present in the silk-based toroidal disc scaffold, the biocompatible gel, or both. The term “cells” used herein generally refers to any eukaryotic cells, e.g., mammalian cells. In some embodiments, the cells can be mammalian cells. Mammalian cells include, without limitation; primate, human and a cell from any animal of interest, including without limitation; mouse, rat, hamster, rabbit, dog, cat, domestic animals, such as equine, bovine, murine, ovine, canine, feline, etc. The cells can be derived from a wide variety of tissue types present in a native intervertebral disc. Stem cells, embryonic stem (ES) cells, ES-derived cells and stem cell progenitors are also included, including without limitation, hematopoietic, neural, stromal, muscle, cardiovascular, hepatic, pulmonary, and gastrointestinal stem cells and adipose-derived stem cells.

In some embodiments, cells seeded in an implantable intervertebral disc device described herein (e.g., a silk-based toroidal disc scaffold, a biocompatible gel, or both) can comprise human stem cells such as, e.g., mesenchymal stem cells, induced pluripotent stem cells (iPSCs), synovial derived stem cells, embryonic stem cells, adult stem cells, umbilical cord blood cells, umbilical Wharton's jelly cells, osteocytes, fibroblasts, neuronal cells, lipocytes, adipocytes, bone marrow cells, assorted immunocytes, precursor cells derived from adipose tissue, bone marrow derived progenitor cells, peripheral blood progenitor cells, stem cells isolated from adult tissue and genetically transformed cells or combinations of the above cells; or differentiated cells such as, e.g., muscle cells, adipose cells.

Stem cells can be obtained with minimally invasive procedures from bone marrow, adipose tissue, or other sources in the body, are highly expandable in culture, and can be readily induced to differentiate into different progenitor cells after exposure to a well-established differentiation supplement.

In some embodiments, cells seeded in an implantable intervertebral disc device described herein (e.g., a silk-based toroidal disc scaffold, a biocompatible gel, or both) can comprise annulus fibrosus (AF) cells. In one embodiment, at least one annulus fibrosus (AF) cell can be present in the interior space of the silk-based toroidal disc scaffold and/or the laminar silk scaffold strips disposed therein. In some embodiments, at least one AF cell can be present along a wall of a laminar porous structure present in a lamellar silk scaffold strip.

The implantable intervertebral disc device described herein can be used to replace, augment or facilitate regeneration of a degenerated intervertebral disc tissue. Accordingly, in some embodiments, the silk-based toroidal disc scaffold can be configured to retain their original volume upon implantation of the implantable intervertebral disc device in a subject for a period of time. As used herein, the term “retain” refers to maintaining the volume (e.g., size and/or shape) of at least a portion of the silk-based toroidal disc scaffold described herein over a period of time. In some embodiments, at least a portion of the silk-based toroidal disc scaffold can retain over a period of time at least about 20% of their original volume, including, for example, at least about 30%, at least about 40%, at least about 50%, at least about 60%, at least about 70%, at least about 80%, at least about 90% of their original volume or higher. In some embodiments, at least a portion of the silk-based toroidal disc scaffold can retain 100% of their original volume, e.g., no detectable changes in the volume, upon implantation to the intervertebral disc tissue to be repaired, augmented, or replaced for a period of time. The volume of the silk-based toroidal disc scaffold placed into a tissue can be determined or indicated by a change in at least one of the tissue properties, e.g., tissue volume, tissue elasticity, and/or tissue hardness. In some embodiments, the volume of the silk-based toroidal disc scaffold placed into a tissue can be determined from explants.

The silk-based toroidal disc scaffold can retain at least a portion of their original volume for any period of time, e.g., weeks, months, or years. In some embodiments, the silk-based toroidal disc scaffold can retain, e.g., at least about 30% of their original volume (including e.g., at least about 40%, at least about 50%, at least about 60%, at least about 70%, at least about 80%, or higher, of their original volume) for at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least 3 years, at least about 4 years, at least 5 years, at least about 10 years or longer.

The volume retention of the silk-based toroidal disc scaffold can also be characterized by, e.g., degradation of the silk-based toroidal disc scaffold. Generally, the slower the silk-based toroidal disc scaffold degrade, the longer the silk-based toroidal disc scaffold can retain their original volume in a tissue. As used in reference to the silk-based toroidal disc scaffold described herein, the term “degrade” or “degradation” refers to a decrease in volume or size of the silk-based toroidal disc scaffold. The degradation of the silk-based toroidal disc scaffold can occur via cleavage of the silk-based toroidal disc scaffold into smaller fragments and/or dissolution of the silk-based toroidal disc scaffold or fragments thereof. In some embodiments, at least a portion of the silk-based toroidal disc scaffold can be adapted to degrade no more than 80% of their original volume, including, for example, no more than 70%, no more than 60%, no more than 50%, no more than 40%, no more than 30%, no more than 20%, no more than 10% of their original volume or lower. In some embodiments, at least a portion of the silk-based toroidal disc scaffold can exhibit no significant degradation (e.g., no detectable changes in the volume) within the intervertebral disc tissue to be repaired, augmented or replaced.

The silk-based toroidal disc scaffold can be adapted to degrade at any rate. In some embodiments, the silk-based toroidal disc scaffold can be adapted to degrade at least a portion of their original volume over any period of time, e.g., weeks, months, or years. In some embodiments, the silk-based toroidal disc scaffold can be adapted to degrade, e.g., no more than 50% of their original volume (including e.g., no more than 40%, no more than 30%, no more than 20% or lower, of their original volume) in at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 10 years or longer.

The same or similar compositions of the silk-based toroidal disc scaffold can manifest different responses in a subject. By way of example only, the volume retention or degradation rate of the silk-based toroidal disc scaffold in an intervertebral disc tissue can vary from one subject to another, e.g., because of different tissue microenvironment such as species and/or levels of various proteins or enzymes (e.g., proteolytic enzymes) present in the intervertebral disc tissue.

The degradation properties of the silk-based toroidal disc scaffold can be modulated, e.g., in part by the beta-sheet content, pore morphologies and architecture, silk processing methods, and/or silk solution concentration described herein. In some embodiments, the silk-based toroidal disc scaffold can comprise a beta sheet content of about 10% to about 90%, or about 15% to about 60%, or about 15% to about 50%.

In some embodiments, the beta sheet content, the porosity of a silk matrix, and/or the dimensions of the lamellar porous structures can be adjusted to yield a resulting silk-based toroidal disc scaffold with mechanical strength comparable to (e.g., within 20%, within 10%, or within 5%) that of a native annulus fibrosus (AF) tissue, e.g., in a subject. For example, in some embodiments, the silk-based toroidal disc scaffold described herein (with or without cells) can have a linear elastic modulus of about 0.05 MPa to about 1 MPa, or about 0.1 MPa to about 0.5 MPa. In some embodiments, the silk-based toroidal disc scaffold described herein (with or without cells) can have an ultimate tensile strength of 0.005 MPa to about 6 MPa, about 0.01 MPa to about 5 MPa, about 0.1 MPa to about 4.5 MPa, or about 0.5 MPa to about 4 MPa. In some embodiments, the silk-based toroidal disc scaffold described herein (with or without cells) can have an elongation to failure of about 10% to about 80% strain, or about 20% to about 60% strain. In some embodiments, the mechanical strength of the silk-based toroidal disc scaffold can increase over time, as the cells seeded therein proliferate and deposit extracellular matrix such as collagen I, and glycosaminoglycans (GAGs) that can contribute to the mechanical strength of the silk-based toroidal disc scaffold.

### Methods for Producing Implantable Intervertebral Disc Devices Described Herein

While implantable intervertebral disc devices described herein can be produced by any known methods in the art, in some embodiments, the implantable intervertebral disc devices can be produced by one or more embodiments of the production method described below. Accordingly, another aspect provided herein relates to methods of producing an implantable intervertebral disc device. In one embodiment, the method of producing an implantable intervertebral disc device described herein comprises: (a) providing a first laminar silk scaffold strip described herein and a second laminar silk scaffold strip described herein; and (b) layering the first laminar silk scaffold strip and the second laminar silk scaffold strip to form a laminate composite, wherein the second laminar silk scaffold strip is positioned such that the alignment axis of the first laminar scaffold strip forms an angle α with the alignment axis of the second laminar scaffold strip, wherein the laminate composite forms a circumferential surface of the toroidal disc scaffold.

As shown in FIG. 14, the angle α defined by the alignment axes 1402A, 1402B on any consecutive laminar scaffold strips (e.g., 1406, 1408) can be in the range from about 20° to about 160°, or from about 30° to about 140°, or from about 35° to about 120°, or from about 40° to about 100°, or from about 40° to about 80°, or from about 50° to about 70°. In some embodiments, the angle α defined by the alignment axes 1402A, 1402B on any consecutive laminar scaffold strips (e.g., 1406, 1408) can be in the range from about 50° to about 70°. In one embodiment, the angle cc defined by the alignment axes 1402A, 1402B on any consecutive laminar scaffold strips (e.g., 1406, 1408) is about 60°.

Stated another way, in various embodiments, at least two laminar silk scaffold strips can be arranged such that the alignment axis 1402B of the first laminar scaffold strip 1406 forms a complementary angle (180°-α) with the alignment axis 1402A of the second laminar silk scaffold strip 1408. The complementary angle (180°-α) defined by the alignment axes 1402A, 1402B on any consecutive laminar scaffold strips (e.g., 1406, 1408) can be in the range from about 20° to about 160°, about 40° to about 150°, about 60° to about 145°, about 80° to about 140°, about 100° to about 140°, or about 110° to about 130°. In some embodiments, the complementary angle (180°-α) defined by the alignment axes 1402A, 1402B on any consecutive laminar scaffold strips (e.g., 1406, 1408) can be in the range from about 110° to about 130°. In some embodiments, the complementary angle (180°-α) defined by the alignment axes 1402A, 1402B on any consecutive laminar scaffold strips (e.g., 1406, 1408) is about 120°.

While in some embodiments, the first laminar silk scaffold strip and the second laminar silk scaffold strip can form a laminate composite prior to forming a circumferential surface of a toroidal disc scaffold, the first laminar silk scaffold strip and the second laminar silk scaffold strip can, in alternative embodiments, sequentially wrap around the same vertical axis to form a circumferential surface of a toroidal disc scaffold while forming a laminate composite. Accordingly, in alternative embodiments, as shown in FIGS. 13 and 14, the method of producing an implantable intervertebral disc device can comprise: (a) providing a first laminar silk scaffold strip described herein (e.g., 1306, 1406) and a second laminar silk scaffold strip described herein (e.g., 1308, 1408); (b) wrapping with a first laminar silk scaffold strip 1306, 1406 circumferentially around a vertical axis 1310; (c) wrapping with a second laminar silk scaffold strip 1308, 1408 circumferentially around the first laminar silk scaffold strip 1306, 1406 in a manner such that the laminar porous structures of the second laminar silk scaffold strip 1308, 1408 are substantially oriented at an angle α of about 20° to about 160° (or at a complementary angle (180°-α) of about 20° to about 160° with respect to the laminar porous structures of the first laminar silk scaffold strip 1306, 1406, thereby producing an implantable intervertebral disc device 1400 comprising a silk-based toroidal disc scaffold 1401 formed from the first 1306, 1406 and the second 1308, 1408 laminar silk scaffold strips.

In some embodiments, as shown in FIG. 13, the method can further comprise wrapping with a third laminar silk scaffold strip 1314 circumferentially around the second laminar silk scaffold strip 1308 in a manner such that the laminar porous structures of the third laminar silk scaffold strip 1314 are substantially oriented at an angle α of about 20° to about 160° (or at a complementary angle (180°-α) of about 20° to about 160° with respect to the laminar porous structures of the second laminar silk scaffold strip 1308. Additional laminar silk scaffold strips can be circumferentially wrapped around one another at an alternating direction to create cross alignment of the lamellar porous structures between each laminar silk scaffold strips.

In some embodiments, the laminate composite or the first laminar silk scaffold strip 1306 can be circumferentially wrapped around a substantially circular disc element 1312 having the vertical axis 1310. In some embodiments, the substantially circular disc element 1312 can comprise a biocompatible gel or hydrogel. The biocompatible or hydrogel can be fabricated from any biocompatible materials or polymer. The biocompatible polymer that can be used herein include, but are not limited to, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, hyaluronic acid and other biocompatible and/or biodegradable polymers. See, e.g., International Application Nos.: WO 04/062697; WO 05/012606, the contents of which are incorporated herein by reference.

In one embodiment, the biocompatible gel or hydrogel can comprise silk fibroin. Methods of producing a silk gel or hydrogel are known in the art, e.g., subjecting a silk solution to a condition comprising freezing, drying (e.g., gas-drying), sonication, shear force, electric field, a pH decrease, or any combinations thereof. Additional details on forming a silk gel from a silk solution can be found, e.g., in U.S. Pat. Nos. 7,674,882; 8,071,722; 7,635,755; 7,842,780; 8,187,616; and International Appl. Nos. WO 2010/087823; WO2011/005381; WO2010/036992, the contents of which are incorporated herein by reference. In other embodiments, the silk gel or hydrogel can further comprise at least one or more biocompatible and/or biodegradable polymers described herein.

In some embodiments, the method can further comprise seeding at least one or more cells into at least a portion of an intervertebral disc device described herein. In some embodiments, the method can further comprise culturing at least one or more cell seeded into at least a portion of an intervertebral disc device described herein.

Production of Porous Structures in a Silk-Based Matrix:

In some embodiments, the silk-based toroidal disc scaffold and/or nucleus pulposus-mimic silk gel described herein can comprise porous structures, e.g., to mimic the structural morphology of a native tissue, to modulate the degradation rate/volume retention rate, and/or to module release profile of an active agent embedded therein, if any. Methods for generating porous structures within a silk fibroin matrix, e.g., freeze-drying, porogen-leaching method (e.g., salt-leaching), and gas foaming methods, are well known in the art and have been described in, e.g., U.S. Pat. No. 7,842,780; and US Patent Application Nos: US 2010/0279112; and US 2010/0279112, the contents of which are incorporated herein by reference.

In some embodiments, porous silk matrices can be produced by freeze-drying method. See, e.g., U.S. Pat. No. 7,842,780, and US 2010/0279112, the contents of which are incorporated herein by reference. The size, shape, and/or orientation of pores as well as porosity within a laminar silk scaffold and/or a nucleus pulposus-mimic silk gel can vary with the freezing process parameters, e.g., freezing rate, types of freezing agent, direction of freezing, and any combinations thereof. Typically, ice crystals that formed in the silk solution can act as porogens and determine the features of the pores once the frozen silk matrix is lyophilized.

The silk fibroin solution can be frozen at any sub-zero temperatures, e.g., depending on selection of a freezing agent, to form a lamellar silk scaffold and/or nucleus pulposus-mimic silk gel. In some embodiments, the silk solution can be frozen at a temperature of less than −15° C. In some embodiments, the silk solution can be frozen at a temperature of about −80° C. to about −20° C. In some embodiments, the silk solution can be frozen at a temperature of less than −50° C. or lower, including, e.g., less than −75° C., or less than −80° C., or less than −100° C., or less than −150° C., or less than −175° C., or less than −200° C.

Without wishing to be bound by theory, pore shape and/or orientation can be controlled by freezing direction. For example, in some embodiments, the silk solution can be subjected to isotropic freezing thereby forming randomly-oriented pores (e.g., interconnected round pores) in a silk matrix. In one embodiment, the silk solution can be isotropically frozen at about −20° C. In another embodiment, the silk solution can be isotropically frozen at about −80° C.

Exemplary Methods of Producing Laminar Silk Scaffold Strips Described Herein:

Laminar silk scaffold strips described herein can be produced by any methods known in the art. In one embodiment, a laminar silk scaffold strip is produced by a method comprising: exposing a silk fibroin solution to unidirectional freezing; and lyophilizing the frozen silk fibroin solution, thereby forming a laminar silk scaffold.

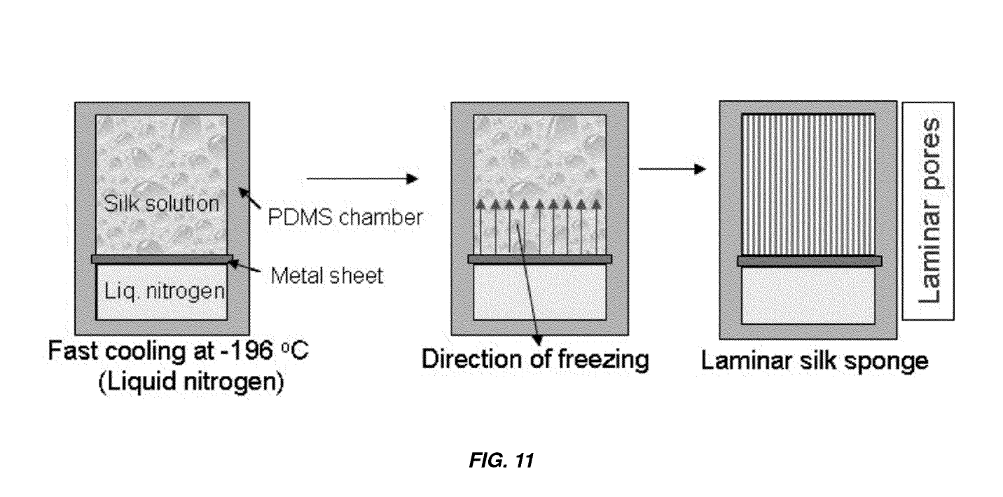

In order to produce lamellar porous structures in a silk matrix (e.g., to form a lamellar silk scaffold), the silk solution can be subjected to unidirectional freezing thereby forming substantially aligned or parallel pores (e.g., as shown in FIGS. 2A, 4A, and 12A) in a silk matrix. In such embodiments, the unidirectional freezing can be performed by a method comprising creating a temperature gradient across the silk solution in one direction, wherein the temperature gradient direction determines the orientation of the pores present in the silk matrix. For example, the temperature gradient can be formed by exposing a portion of the silk solution to a freezing agent, e.g., without limitations, liquid nitrogen, a mixture of dry ice and alcohol, or a combination thereof. In one embodiment, the silk solution can be subjected to unidirectional freezing in a custom-designed PDMS mold (e.g., as shown in FIGS. 10 and 11), e.g., using the protocol described in the Examples.

The freezing rate of the silk solution can affect the pore size formed in the lamellar silk matrix scaffold. Without wishing to be bound by theory, the pore size typically increases with a slower freezing rate, because a slower freezing rate generally result in larger ice crystal and in turn larger pores. In some embodiments, the freezing rate (e.g., the distance travelled by the freezing front over a period of time) can range from about 0.05 mm/min to about 10 mm/min, 0.1 mm/min to about 5 mm/min, or 0.5 mm/min to about 3 mm/min. A skilled artisan can optimize the freezing rate (e.g., by using different freezing agents) to form pores of a desirable size. For example, in one embodiment, liquid nitrogen can be used to freeze a silk solution at a rate of about 2.5 mm/min to about 3.5 mm/min (e.g., ˜3 mm/min). In another embodiment, absolute ethanol and dry ice bath can be used to freeze a silk solution at a rate of about 0.5 mm/min to about 1 mm/min (e.g., ˜0.7 mm/min). In another embodiment, less than 100% v/v ethanol (e.g., ˜70% v/v) and dry ice bath can be used to freeze a silk solution at a rate of about 0.1 mm/min to about 0.5 mm/min (e.g., ˜0.4 mm/min).

In some embodiments, the frozen silk fibroin solution after unidirectional freezing can be lyophilized for at least about 12 hours, at least about 24 hours, at least about 2 days, at least about 3 days or more.

In some embodiments, after formation of the solid-state silk matrix (e.g., a lamellar silk scaffold and/or a nucleus pulposus-mimic silk gel), the method can further comprise exposing the solid-state silk matrix to a post-treatment that will affect at least one silk fibroin property. For example, post-treatment of a silk matrix can affect silk fibroin properties including beta-sheet content, solubility, active agent loading capacity, degradation time, active agent permeability or any combinations thereof. Silk post-processing options, e.g., to increase beta-sheet content, include, but not limited to, controlled slow drying (Lu et al., 10 Biomacromolecules 1032 (2009)), water annealing (Jin et al., Water-Stable Silk Films with Reduced β-Sheet Content, 15 Adv. Funct. Mats. 1241 (2005); Hu et al. Regulation of Silk Material Structure by Temperature-Controlled Water Vapor Annealing, 12 Biomacromolecules 1686 (2011)), stretching (Demura & Asakura, Immobilization of glucose oxidase with Bombyx mori silk fibroin by only stretching treatment and its application to glucose sensor, 33 Biotech & Bioengin. 598 (1989)), compressing, and solvent immersion, including methanol (Hofmann et al., 2006), ethanol (Miyairi et al., 1978), glutaraldehyde (Acharya et al., 2008) and 1-ethyl-3-(3-dimethyl aminopropyl) carbodiimide (EDC) (Bayraktar et al., 2005); pH adjustment (see, e.g., U.S. Patent App. No. US2011/0171239, the content of which is incorporated herein by reference), heat treatment, shear stress (see, e.g., International App. No.: WO 2011/005381, the content of which is incorporated herein by reference), sonication (see, e.g., U.S. Pat. App. No. U.S. 2010/0178304 and International App. No.: WO 2008/150861, the contents of which are incorporated herein by reference), and any combinations thereof.

In some embodiments, post-treatment of a silk matrix can increase beta-sheet content in the silk matrix, e.g., by water annealing or solvent immersion such as methanol and/or ethanol. In some embodiments, post-treatment of a silk matrix, e.g., water-annealing or solvent immersion, can modulate the degradation or solubility properties of the silk matrix (e.g., upon in vivo implantation). In some embodiments, post-treatment of a solid-state silk matrix, e.g., water-annealing or solvent immersion, can control the release of an active agent, if any, from the silk matrix.

In some embodiments, the silk fibroin solution to be subjected to unidirectional freezing for production of a laminar silk scaffold strip can further comprise a water-soluble pore-forming agent, e.g., but not limited to, sodium alginate, or other equivalent water-soluble particles. The addition of these water-soluble pore-forming agents into a silk fibroin solution can generate larger inter-lamellar distance, due to different ice crystal sizes during freezing. In one embodiment, about 0.05%-1% (e.g., ˜0.2%) sodium alginate can be mixed into a silk solution.

In some embodiments where a water-soluble pore-forming agent is added, the method of producing a laminar silk scaffold strip can further comprise removing the water-soluble pore-forming agent from the formed laminar silk scaffold. For example, the formed laminar silk scaffold can be submerged in water for sufficient time (e.g., at least about 6 hours, at least about 12 hours or more) to remove the water-soluble pore-forming agent therein.

In some embodiments, the method of producing a laminar silk scaffold strip can further comprise reducing the laminar silk scaffold (e.g. if the scaffold dimension is larger than a dimension of an annulus fibrosus) into strips of smaller dimensions, e.g., the first laminar silk scaffold strip and the second laminar silk scaffold strip. For example, as shown in FIG. 13, thin strips 1306 or 1308 (e.g., ˜1 cm wide or less) can be cut from a laminar silk scaffold 1300 produced using the method described herein such that at least about 5% or more (including, e.g., at least about 10%, at least about 20%, at least about 30%, at least about 40%, at least about 50%, at least about 75%, at least about 90% or more) of the lamellar porous structures are substantially aligned to an alignment axis 1302, e.g., forming a pre-determined angle θ (e.g., in one embodiment, the pre-determined angle θ is about 30° with a reference axis such as a transverse axis 1303 of the strip 1306 or 1308.

Silk Fibroin and Silk Solutions:

Silk fibroin is a particularly appealing biopolymer for use in various embodiments described herein, e.g., because of its versatile processing e.g., all-aqueous processing (Sofia et al., 54 J. Biomed. Mater. Res. 139 (2001); Perry et al., 20 Adv. Mater. 3070-72 (2008)), relatively easy functionalization (Murphy et al., 29 Biomat. 2829-38 (2008)), biocompatibility, biodegradability and tough material properties, as well as its ability to be reprocessed into various formats (Santin et al., 46 J. Biomed. Mater. Res. 382-9 (1999); Vepari and Kaplan 2007). For example, silk has been approved by U.S. Food and Drug Administration as a tissue engineering scaffold in human implants. See Altman et al., 24 Biomaterials: 401 (2003). While silk porous scaffolds have been previously reported for use in annulus fibrosus (AF) tissue engineering (Chang et al., 2007; and Change et al., 2010), the lamellar AF structure was not mimicked in those AF scaffolds.

As used herein, the term “silk fibroin” includes silkworm fibroin and insect or spider silk protein. See e.g., Lucas et al., 13 Adv. Protein Chem. 107 (1958). Any type of silk fibroin including naturally-derived synthetic silk fibroin may be used according to various aspects described herein. Silk fibroin produced by silkworms, such as Bombyx mori, is the most common and represents an earth-friendly, renewable resource. For instance, silk fibroin may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants (see, e.g., WO 97/08315; U.S. Pat. No. 5,245,012), and variants thereof, that can be used. In some embodiments, silk fibroin for use in the methods described herein can be modified to comprise an active agent or be conjugated to an active agent.

The silk-based toroidal disc scaffold and/or the silk gel (to mimic nucleus pulposus) can be produced from aqueous-based or organic solvent-based silk fibroin solutions. In some embodiments, the silk-based toroidal disc scaffold and/or the silk gel (to mimic nucleus pulposus) produced from organic solvent-based silk fibroin solution can retain their original volume for a longer period of time than the aqueous-based silk-based toroidal disc scaffold and/or the silk gel (to mimic nucleus pulposus). The aqueous- or organic solvent-based silk fibroin solution used for making laminar silk scaffold strips described herein can be prepared using any techniques known in the art. The concentration of silk fibroin in solutions used for producing the laminar silk scaffold strips can be suited to needs. By way of example only, for intervertebral disc (IVD) repair, augmentation or replacement, the concentration of silk fibroin in solutions can be suited to a particular degradation profile, e.g., higher concentrations of silk fibroin solutions can be used when higher resistance to degradation is desired for the IVD repair, augmentation or replacement. In some embodiments, the silk fibroin solution for making the laminar silk scaffold strips can vary from about 0.1% (w/v) to about 30% (w/v), inclusive. In some embodiments, the silk fibroin solution can vary from about 0.5% (w/v) to about 15% (w/v). In some embodiments, the silk fibroin solution can vary from about 1% (w/v) to about 10% (w/v). In some embodiments, the silk fibroin solution can vary from about 5% (w/v) to about 10% (w/v). Suitable processes for preparing silk fibroin solution are disclosed, for example, in U.S. Pat. No. 7,635,755; and International Application Nos: WO/2005/012606; and WO/2008/127401. A micro-filtration step can be used herein. For example, the prepared silk fibroin solution can be processed further, e.g., by centrifugation and/or syringe based micro-filtration before further processing into silk fibroin-based matrices described herein.

In various embodiments, the silk fibroin can be modified for different applications and/or desired mechanical or chemical properties (e.g., to facilitate formation of a gradient of active agent in a silk fibroin matrix, e.g., a silk-based toroidal disc scaffold, a laminar silk scaffold strip, and/or a nucleus pulposus-mimic silk gel). One of skill in the art can select appropriate methods to modify silk fibroins, e.g., depending on the side groups of the silk fibroins, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g. U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142). Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. Additionally, the silk fibroin matrix can be combined with a chemical, such as glycerol, that, e.g., affects flexibility of the matrix. See, e.g., WO 2010/042798, Modified Silk films Containing Glycerol.

In some embodiments, the silk fibroin can be also mixed with other biocompatible and/or biodegradable polymers to form mixed polymer matrices comprising silk fibroin. One or more biocompatible and/or biodegradable polymers (e.g., two or more biocompatible polymers) can be added to the silk fibroin solution. The biocompatible polymer that can be used herein include, but are not limited to, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, hyaluronic acid and other biocompatible and/or biodegradable polymers. See, e.g., International Application Nos.: WO 04/062697; WO 05/012606, the contents of which are incorporated herein by reference.

In some embodiments, at least one active agent described herein can be added to the silk fibroin solution before further processing into a silk matrix described herein, e.g., a silk-based toroidal disc scaffold, laminar silk scaffold strips and/or a nucleus pulposus-mimic silk gel. In some embodiments, the active agent can be dispersed homogeneously or heterogeneously within the silk fibroin, dispersed in a gradient, e.g., using the carbodiimide-mediated modification method described in the U.S. Patent Application No. US 2007/0212730.

In some embodiments, the silk matrix described herein, e.g., a silk-based toroidal disc scaffold, laminar silk scaffold strips and/or a nucleus pulposus-mimic silk gel can be first formed and then contacted with (e.g., dipped into) at least one active agent such that the active agent can coat the open surface of the silk matrix or penetrate into the pores of the silk matrix.

Further, the solid-state silk matrices described herein (e.g., a silk-based toroidal disc scaffold, laminar silk scaffold strips, or a nucleus pulposus-mimic silk gel) can take advantage of the many techniques developed to functionalize silk matrix or silk fibroin (e.g., active agents such as dyes and sensors). See, e.g., U.S. Pat. No. 6,287,340, Bioengineered anterior cruciate ligament; WO 2004/000915, Silk Biomaterials & Methods of Use Thereof; WO 2004/001103, Silk Biomaterials & Methods of Use Thereof; WO 2004/062697, Silk Fibroin Materials & Use Thereof; WO 2005/000483, Method for Forming inorganic Coatings; WO 2005/012606, Concentrated Aqueous Silk Fibroin Solution & Use Thereof; WO 2011/005381, Vortex-Induced Silk fibroin Gelation for Encapsulation & Delivery; WO 2005/123114, Silk-Based Drug Delivery System; WO 2006/076711, Fibrous Protein Fusions & Uses Thereof in the Formation of Advanced Organic/Inorganic Composite Materials; U.S. Application Pub. No. 2007/0212730, Covalently immobilized protein gradients in three-dimensional porous scaffolds; WO 2006/042287, Method for Producing Biomaterial Scaffolds; WO 2007/016524, Method for Stepwise Deposition of Silk Fibroin Coatings; WO 2008/085904, Biodegradable Electronic Devices; WO 2008/118133, Silk Microspheres for Encapsulation & Controlled Release; WO 2008/108838, Microfluidic Devices & Methods for Fabricating Same; WO 2008/127404, Nanopatterned Biopolymer Device & Method of Manufacturing Same; WO 2008/118211, Biopolymer Photonic Crystals & Method of Manufacturing Same; WO 2008/127402, Biopolymer Sensor & Method of Manufacturing Same; WO 2008/127403, Biopolymer Optofluidic Device & Method of Manufacturing the Same; WO 2008/127401, Biopolymer Optical Wave Guide & Method of Manufacturing Same; WO 2008/140562, Biopolymer Sensor & Method of Manufacturing Same; WO 2008/127405, Microfluidic Device with Cylindrical Microchannel & Method for Fabricating Same; WO 2008/106485, Tissue-Engineered Silk Organs; WO 2008/140562, Electroactive Biopolymer Optical & Electro-Optical Devices & Method of Manufacturing Same; WO 2008/150861, Method for Silk Fibroin Gelation Using Sonication; WO 2007/103442, Biocompatible Scaffolds & Adipose-Derived Stem Cells; WO 2009/155397, Edible Holographic Silk Products; WO 2009/100280, 3-Dimensional Silk Hydroxyapatite Compositions; WO 2009/061823, Fabrication of Silk Fibroin Photonic Structures by Nanocontact Imprinting; WO 2009/126689, System & Method for Making Biomaterial Structures.

In alternative embodiments, the implantable intervertebral disc device can include plasmonic nanoparticles (e.g., but not limited to, gold nanoparticles) to form photothermal elements. For example, plasmonic nanoparticles can be added to a silk solution to form a silk-based toroidal disc scaffold, laminar silk scaffold strips and/or nucleus pulposus-mimic silk gel. This approach takes advantage of the superior doping characteristics of silk fibroin. Thermal therapy has been shown to aid in the delivery of various agents, see Park et al., Effect of Heat on Skin Permeability, 359 Intl. J. Pharm. 94 (2008). In one embodiment, short bursts of heat on very limited areas can be used to maximize permeability with minimal harmful effects on surrounding tissues. Thus, plasmonic particle-doped silk matrices can add specificity to thermal therapy by focusing light to locally generate heat only via the silk matrices. In some embodiments, the silk matrices can include photothermal agents such as gold nanoparticles.

In some embodiments, the solid-state silk matrix (e.g., a silk-based toroidal disc scaffold, laminar silk scaffold strips, and/or silk gel) can be coated with at least one layer of a biocompatible and/or biodegradable polymer described herein, e.g., to modulate the degradation and/or volume retention properties of the implantable intervertebral disc device upon implantation to repair, replace or facilitate regeneration of a degenerated intervertebral disc device and/or to modulate the rate of active agents released from the solid-state silk matrices. In such embodiments, the biocompatible and/or biodegradable polymer can comprise at least one active agent.

In some embodiments, the solid-state silk matrix (e.g., a silk-based toroidal disc scaffold, laminar silk scaffold strips, and/or silk gel) described herein can be coated with cell adhesion molecules, e.g., but not limited to, fibronectin, vitronectin, laminin, collagen, any art-recognized extracellular matrix molecules, and any combinations thereof.

In some embodiments, the implantable intervertebral disc device and/or the solid-state silk matrix (e.g., a silk-based toroidal disc scaffold, laminar silk scaffold strips, and/or silk gel) described herein can be sterilized. Sterilization methods for biomedical devices are well known in the art, including, but not limited to, gamma or ultraviolet radiation, autoclaving (e.g., heat/steam); alcohol sterilization (e.g., ethanol and methanol); and gas sterilization (e.g., ethylene oxide sterilization).

### Exemplary Active Agents

In some embodiments, the implantable intervertebral disc device described herein can further comprise at least one active agent. The active agent can be mixed, or dispersed in the implantable intervertebral disc device, and/or it can be distributed or embedded in the silk-based toroidal disc scaffold, a laminar silk scaffold strip and/or a nucleus pulposus-mimic biocompatible gel. In some embodiments, the active agent can be distributed, embedded or encapsulated in the silk-based toroidal disc scaffold, a laminar silk scaffold strip and/or a nucleus pulposus-mimic biocompatible gel. In some embodiments, the active agent can be coated on surfaces of the silk-based toroidal disc scaffold, a laminar silk scaffold strip and/or a nucleus pulposus-mimic biocompatible gel. The term “active agent” can also encompass combinations or mixtures of two or more active agents, as described below. Examples of an active agent can include, but are not limited to, a cell, a therapeutic agent, an anesthetic, a cell growth factor, a peptide, a peptidomimetic, an antibody or a portion thereof, an antibody-like molecule, nucleic acid (e.g., but not limited to, DNA, RNA, siRNA, shRNA), a polysaccharide, an aptamer, an enzyme, a receptor antagonist or agonist, a hormone, an autogenous bone marrow, an antibiotic, an antimicrobial agent, a small molecule, a cell attachment agent, a macrophage-skewing agent or any combinations thereof. In some embodiments, the active agent(s) can also include, without limitations, anti-inflammatory agents, anesthetics, active agents that stimulate issue formation, and/or healing and regrowth of natural tissues, and any combinations thereof.

Anti-inflammatory agents can include, but are not limited to, naproxen, sulindac, tolmetin, ketorolac, celecoxib, ibuprofen, diclofenac, acetylsalicylic acid, nabumetone, etodolac, indomethacin, piroxicam, cox-2 inhibitors, ketoprofen, antiplatelet medications, salsalate, valdecoxib, oxaprozin, diflunisal, flurbiprofen, corticosteroids, MMP inhibitors and leukotriene modifiers or combinations thereof.

Agents that increase formation of new tissues and/or stimulates healing or regrowth of native tissue can include, but are not limited to, fibroblast growth factor (FGF), transforming growth factor-beta (TGF-β, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof.

Anesthetics can include, but are not limited to, those used in caudal, epidural, inhalation, injectable, retrobulbar, and spinal applications, such as bupivacaine, lidocaine, benzocaine, cetacaine, ropivacaine, and tetracaine, or combinations thereof.

The term “cells” used herein generally refers to any eukaryotic cells, e.g., mammalian cells. In some embodiments, the cells can be mammalian cells. Mammalian cells include, without limitation; primate, human and a cell from any animal of interest, including without limitation; mouse, rat, hamster, rabbit, dog, cat, domestic animals, such as equine, bovine, murine, ovine, canine, feline, etc. The cells can be derived from a wide variety of tissue types present in a native intervertebral disc. Stem cells, embryonic stem (ES) cells, ES-derived cells and stem cell progenitors are also included, including without limitation, hematopoietic, neural, stromal, muscle, cardiovascular, hepatic, pulmonary, and gastrointestinal stem cells and adipose-derived stem cells. In some embodiments, cells seeded in an implantable intervertebral disc device described herein (e.g., a silk-based toroidal disc scaffold, a biocompatible gel, or both) can comprise human stem cells such as, e.g., mesenchymal stem cells, induced pluripotent stem cells (iPSCs), synovial derived stem cells, embryonic stem cells, adult stem cells, umbilical cord blood cells, umbilical Wharton's jelly cells, osteocytes, fibroblasts, neuronal cells, lipocytes, adipocytes, bone marrow cells, assorted immunocytes, precursor cells derived from adipose tissue, bone marrow derived progenitor cells, peripheral blood progenitor cells, stem cells isolated from adult tissue and genetically transformed cells or combinations of the above cells; or differentiated cells such as, e.g., muscle cells, adipose cells.

Stem cells can be obtained with minimally invasive procedures from bone marrow, adipose tissue, or other sources in the body, are highly expandable in culture, and can be readily induced to differentiate into different progenitor cells after exposure to a well-established differentiation supplement. Cells can be obtained from donors (allogenic) or from recipients (autologous). Cells can also be of established cell culture lines, or even cells that have undergone genetic engineering. Additionally, cells can be collected from a multitude of hosts including but not limited to human autograft tissues, transgenic mammals, or bacterial cultures (possibly for use as a probiotic treatment).

In some embodiments, the cells seeded in an implantable intervertebral disc device described herein (e.g., a silk-based toroidal disc scaffold, a biocompatible gel, or both) can comprise annulus fibrosus (AF) cells. In some embodiments, the cells seeded in an implantable intervertebral disc device described herein (e.g., a silk-based toroidal disc scaffold, a biocompatible gel, or both) can comprise annulus fibrosus (AF) cells harvested from a subject whose intervertebral disc is degenerated and needs to be repaired, regenerated or replaced.

In some embodiments, the active agents can be cell attachment agents. Examples of cell attachment agents include, but are not limited to, hyaluronic acid, collagen, crosslinked hyaluronic acid/collagen, an integrin-binding molecule, chitosan, elastin, fibronectin, vitronectin, laminin, proteoglycans, any derivatives thereof, and any combinations thereof.

In some embodiments, the implantable intervertebral disc device described herein (e.g., a silk-based toroidal disc scaffold, a biocompatible gel, or both) can comprise metallic nanoparticles (e.g., but not limited to, gold nanoparticles), optical molecules (e.g., but not limited to, fluorescent molecules, and/or quantum dots), and any other art-recognized contrast agent for various purposes, e.g., for biomedical imaging or photothermal therapy.

When the active agents are embedded in the silk fibroin matrix (e.g., a silk-based toroidal disc scaffold, a biocompatible silk gel, or both), the bioactivity of the active agents (e.g., at least about 30% of the bioactivity of the active agents) can be stabilized for a period of time (e.g., days, weeks, or months) under specific conditions. Such conditions can include, but are not limited to, a state-changing cycle (e.g., freeze-thaw cycles), temperatures (e.g., above 0° C.), air pressures, humidity, and light exposure. See WO/2012/145739, the content of which is incorporated herein by reference. Some embodiments of the injectable composition can be stored or transported between about 0° C. and about 60° C., about 10° C. and about 60° C., or about 15° C. and about 60° C. In these embodiments, after the implantable intervertebral disc device has been implanted into a subject, e.g., a human subject with a body temperature of about 37° C., the bioactivity of the active agents (e.g., at least about 30% of the bioactivity of the active agents) can be stabilized for a period of time, e.g., at least about 3 weeks or longer.

### Applications of the Implantable Intervertebral Disc Devices Described Herein

A further aspect provided herein relates to uses of the implantable intervertebral disc devices described herein. For example, one or more embodiments of the implantable intervertebral disc devices described herein can be used for treatment of a degenerated intervertebral disc (IVD) or annulus fibrous in a subject, and/or as a drug delivery device/depot. In some embodiments, the implantable intervertebral disc device implanted into a degenerated IVD or annulus fibrosus to be repaired, augmented or replaced can act as a scaffold to mimic the extracellular matrices (ECM) of the body, and/or promote tissue regeneration. The scaffold can serve as both a physical support and/or an adhesive template for cells to proliferate therein. In some embodiments, the implantable intervertebral disc device can increase differentiation of annulus fibrosus cells, and thus production of AF ECM such as fibrillar collagen and proteoglycans, e.g., aggrecan. In some embodiments, the implantable intervertebral disc device described herein can contain no cells. Yet the implantable intervertebral disc device can be coated with cell attachment agents, e.g., collagen, and/or chemoattractants, e.g., growth factors, that can attract host cells to the implanted intervertebral disc device and support the cell proliferation. In some embodiments, the implantable intervertebral disc device can be seeded with cells (e.g., stem cells and/or IVD-associated cells prior to implantation to a IVD tissue or annulus fibrosus to be repaired or augmented.

As used herein, by the term “augmenting” or “augmentation” is meant increasing, filling in, restoring, enhancing or replacing an IVD tissue or a portion thereof, e.g., an annulus fibrosus. In some embodiments, the tissue can be partially or completely lost (e.g., removal of a tissue) or damaged.

The term “repair” or “repairing” as used herein, with respect to a IVD tissue or a portion thereof, refers to any correction, reinforcement, reconditioning, remedy, regenerating, filling of an IVD tissue or a portion thereof (e.g., annulus fibrosus) that restores volume, shape and/or function of the tissue. In some embodiments “repair” includes full repair and partial repair. For example, the volume, shape and/or function of an IVD tissue or a portion thereof (e.g., annulus fibrosus) to be repaired can be restored by at least about 30%, at least about 40%, at least about 50%, at least about 60%, at least about 70%, at least about 80% or higher, as compared to no treatment. In some embodiments, the volume, shape and/or function of an IVD tissue or a portion thereof (e.g., annulus fibrosus) to be repaired can be restored by at least about 90%, at least about 95%, at least about 97%, or higher, as compared to no treatment. In some embodiments, the volume, shape and/or function of an IVD tissue or a portion thereof (e.g., annulus fibrosus) to be repaired can be restored by 100% (defect-free or the defect is undetectable by one of skill in the art), as compared to no treatment. In some embodiments, the term “repair” or “repairing” are used herein interchangeably with the term “regeneration” or “regenerate” when used in reference to tissue treatment.

In one embodiment, provided herein is a method of treating a disease or disorder associated with degeneration of an intervertebral disc or a portion thereof in a subject, wherein the method comprises replacing the degenerated intervertebral disc or a portion thereof of the subject with one or more embodiments of the implantable intervertebral disc device described herein.

In some embodiments, the method of treatment can further comprising implanting cells prior to, concurrently with, or after implantation of the implantable intervertebral disc device described herein into a degenerated IVD tissue or a portion thereof. A combination implantation of the intervertebral disc device and cells described herein can be used for a biologically enhanced repair. In some embodiments, implanting cells with the implantable intervertebral disc device described herein can enhance or accelerate host integration and/or tissue formation over time. Cells could be collected from a multitude of hosts including but not limited to human autograft tissues, or transgenic mammals. More specifically, human cells used can comprise cells selected from stem cells (e.g., adipocyte-derived or bone-marrow derived stem cells), osteocytes, fibroblasts, lipocytes, assorted immunocytes, annulus fibrosus cells. In some embodiments, the cells can be added into the implantable intervertebral disc device or components thereof (e.g., a silk-based toroidal disc scaffold, a laminar silk scaffold strip, a nucleus pulposus-mimic biocompatible gel) prior to the implantation. Without wishing to be bound by theory, the cells can secrete pro-angiogenic factors and/or growth factors at the implantation site. As the IVD tissue regenerates, the implanted intervertebral disc device can degrade accordingly. In some embodiments, the implanted intervertebral disc device can integrate with the regenerated host tissue.

Any cells described herein can be seeded upon a surface of an implantable intervertebral disc device described herein. For example, an implantable intervertebral disc device described herein can be submersed in an appropriate growth medium for the cells of interest, and then directly exposed to the cells. The cells are allowed to migrate and proliferate on the surface and into interstitial pores of the implantable intervertebral disc device described herein (e.g., within the silk-based toroidal disc scaffold and/or the silk gel). The implantable intervertebral disc device described herein can then be removed from the growth medium, washed if necessary, and administered. Alternatively, the cells can be placed in a suitable buffer or liquid growth medium and drawn through an implantable intervertebral disc device described herein by using vacuum filtration. Cells can also be admixed with silk fibroin solution prior to forming a laminar silk fibroin scaffold and/or silk gel, capturing at least some of the cells therein. In another embodiment, the cells of interest can be dispersed into an appropriate solution (e.g., a growth medium or buffer) and then sprayed onto an implantable intervertebral disc device described herein. For example, electro-spraying involves subjecting a cell-containing solution with an appropriate viscosity and concentration to an electric field sufficient to produce a spray of small charged droplets of solution that contain cells.

In some embodiments, an implantable intervertebral disc device described herein (e.g., a silk-based toroidal disc scaffold, a lamellar silk scaffold strip, and/or a nucleus pulposus-mimic biocompatible gel) comprising at least one active agent can be used as a platform for drug delivery. For example, the implantable intervertebral disc device described herein (e.g., a silk-based toroidal disc scaffold, a lamellar silk scaffold strip, and/or a nucleus pulposus-mimic biocompatible gel) can be formed with a pharmaceutical agent either entrained in or bound to the silk-based toroidal disc scaffold, the lamellar silk scaffold strip, and/or the nucleus pulposus-mimic biocompatible gel, and then implanted into the body.

For extended or sustained release, a silk-based toroidal disc scaffold, a lamellar silk scaffold strip, and/or a nucleus pulposus-mimic biocompatible gel of the implantable intervertebral disc device described herein can be manipulated, e.g., to modulate its beta-sheet content, for its volume retention and/or degradation rate. In some embodiments, an active agent such as a therapeutic agent can be further crosslinked to the silk matrix in order to enhance the stability and extend the release period. In an alternative approach, silk fibroin matrix can be mixed with other polymers, for examples, hyaluronic acid, to prolong the release of certain growth factors or cytokines and to stabilize the functionality.

As used herein, the term “sustained release” refers to the release of a pharmaceutically-active drug over a period of about seven days or more. In some aspects of this embodiment, a drug delivery platform comprising an intervertebral disc device described herein can release a pharmaceutically-active drug over a period of, e.g., at least about 7 days after implantation, at least about 15 days after implantation, at least about 30 days after implantation, at least about 45 days after implantation, at least about 60 days after implantation, at least about 75 days after implantation, at least about 90 days after implantation, at least about 6 months after implantation, or at least about 1 year after implantation.

As used herein, the term “extended release” refers to the release of a pharmaceutically-active drug over a period of time of less than about seven days. In such embodiments, a drug delivery platform comprising an intervertebral disc device described herein can release a pharmaceutically-active drug over a period of, e.g., about 1 day after implantation, about 2 days after implantation, about 3 days after implantation, about 4 days after implantation, about 5 days after implantation, or about 6 days after implantation.

Embodiments of the Various Aspects Described Herein can be Illustrated by the Following Numbered Paragraphs.
1. An implantable intervertebral disc device comprising:
   a silk-based toroidal disc scaffold, the toroidal disc scaffold comprising on its circumferential surface at least two concentric layers of laminar silk scaffold strips;
   wherein a first laminar silk scaffold strip comprises at least about 5% of its first porous structures substantially aligned at a predetermined angle to the bottom edge of the first laminar scaffold strip;
   wherein a second laminar silk scaffold strip circumferentially wraps around the first laminar silk scaffold strip in a manner such that the second laminar silk scaffold strip comprises at least about 5% of its second porous structures substantially aligned at an angle of about 20° to about 160° with respect to the aligned first porous structures.
2. The device of paragraph 1, wherein at least about 10%, at least about 20%, at least about 30%, at least about 40%, at least about 50%, at least about 75%, or at least about 90% of the first porous structures are substantially aligned at the predetermined angle to the bottom edge of the first laminar scaffold strip.
3. The device of paragraph 1 or 2, wherein at least about 10%, at least about 20%, at least about 30%, at least about 40%, at least about 50%, at least about 75%, or at least about 90% of the second porous structures are substantially aligned at the angle of about 20° to about 160° with respect to the aligned first porous structures.
4. The device of any of paragraphs 1-3, wherein said at least two concentric layers includes a range of about 2 to about 30 concentric layers, or about 10 to about 20 concentric layers.
5. The device of any of paragraphs 1-4, wherein at least about 5% of the porous structures between any two successive concentric layers are substantially aligned at an angle of about 80° to about 140° with respect to each other.
6. The device of any of paragraphs 1-5, wherein the predetermined angle ranges from about 10° to about 80°, or about 15° to about 60°, or about 20° to about 40°, or is about 30°.
7. The device of any of paragraphs 1-6, wherein the porous structures have a pore size of about 10 μm to about 500 μm, or about 100 μm to about 250 μm.
8. The device of any of paragraphs 1-7, wherein an inter-lamellar spacing in the lamellar silk scaffold strips ranges from about 10 μm to about 500 μm, or about 150 μm to about 250 μm.
9. The device of any of paragraphs 1-8, further comprising a biocompatible gel surrounded by the silk-based toroidal disc scaffold.
10. The device of paragraph 9, wherein the biocompatible gel comprises silk fibroin.
11. The device of any of paragraphs 1-10, further comprising an active agent.
12. The device of paragraph 11, wherein the active agent is selected from the group consisting of a cell, a therapeutic agent, an anesthetic, a cell growth factor, a peptide, a peptidomimetic, an antibody or a portion thereof, an antibody-like molecule, nucleic acid (e.g., but not limited to, DNA, RNA, siRNA, shRNA), a polysaccharide, an aptamer, an enzyme, a receptor antagonist or agonist, a hormone, an autogenous bone marrow, an antibiotic, an antimicrobial agent, a small molecule, a cell attachment agent, a macrophage-skewing agent or any combinations thereof.
13. The device of paragraph 12, wherein the active agent comprises at least one intervertebral disc cell present in the silk-based toroidal disc scaffold, the biocompatible gel, or both.
14. A method of producing an implantable intervertebral disc device comprising:
   a. providing a first and a second laminar silk scaffold strips, wherein the first and the second laminar silk scaffold strips each comprises at least about 5% of its porous structures substantially aligned at a predetermined angle;
   b. wrapping with a first laminar silk scaffold strip circumferentially around a vertical axis;
   c. wrapping with a second laminar silk scaffold strip circumferentially around the first laminar silk scaffold strip in a manner such that the aligned porous structures of the second laminar silk scaffold strip are substantially oriented at an angle of about 20° to about 160° with respect to the aligned porous structures of the first laminar silk scaffold strip, thereby producing an implantable intervertebral disc device comprising a silk-based toroidal disc scaffold formed from the first and the second laminar silk scaffold strips.
15. The method of paragraph 14, wherein the angle of about 20° to about 160° includes an angle of about 80° to about 140°, or about 110° to about 130°, or an angle of about 120°.
16. The method of paragraph 14 or 15, wherein the laminar silk scaffold strips are produced by a method comprising:
   a. exposing a silk fibroin solution to unidirectional freezing; and
   b. lyophilizing the frozen silk fibroin solution, thereby forming a laminar silk scaffold.
17. The method of paragraph 16, wherein the method of producing the laminar silk scaffold strips further comprises reducing the laminar silk scaffold into the first and the second laminar silk scaffold strips.
18. The method of paragraph 16 or 17, wherein when the silk fibroin solution further comprises a water-soluble pore-forming agent, the method of producing the laminar silk scaffold strips further comprises removing the water-soluble pore-forming agent from the laminar silk scaffold.
19. The method of any of paragraphs 17-18, wherein the water-soluble pore-forming agent includes sodium alginate.
20. The method of any of paragraphs 14-19, further comprising post-treatment of the laminar silk scaffold to increase insolubility of the laminar silk scaffold in an aqueous solution.
21. The method of any of paragraphs 14-20, wherein the wrapping around the vertical axis comprises wrapping with the first laminar silk scaffold strip circumferentially around a substantially circular disc element having the vertical axis.
22. The method of paragraph 21, wherein the substantially circular disc element comprises a biocompatible gel.
23. The method of paragraph 22, wherein the biocompatible gel comprises silk fibroin.
24. The method of any of paragraphs 14-23, further comprising seeding at least one intervertebral disc cell into the device.
25. An implantable intervertebral disc device produced by the method of any of paragraphs 14-24.
26. A method of treating a disease or disorder associated with degeneration of an intervertebral disc in a subject comprising implanting at a target site in a subject an implantable intervertebral disc device of any of paragraphs 1-13.
27. The method of paragraph 26, further comprising replacing the degenerated intervertebral disc of the subject with the implantable intervertebral disc device.
28. The method of paragraph 26 or 27, further placing cells at the target site in the subject.
29. The method of paragraph 28, wherein the cells comprise stem cells and/or intervertebral disc-associated cells.

### Some Selected Definitions of Terms

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate, e.g., a human. A subject can be male or female. Preferably, the subject is a mammal. The mammal can be a human, non-human primate, mouse, rat, dog, cat, horse, or cow, but are not limited to these examples. Mammals other than humans can be advantageously used as subjects that represent animal models of tissue repair, regeneration and/or reconstruction. In addition, the methods and compositions described herein can be used to treat domesticated animals and/or pets.

The term “statistically significant” or “significantly” refers to statistical significance and generally means a two standard deviation (2SD) below or above a reference level. The term refers to statistical evidence that there is a difference. It is defined as the probability of making a decision to reject the null hypothesis when the null hypothesis is actually true. The decision is often made using the p-value.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides.

The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated. The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that can form hairpin structures comprising a duplex region.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA.

The term “enzymes” as used here refers to a protein molecule that catalyzes chemical reactions of other substances without it being destroyed or substantially altered upon completion of the reactions. The term can include naturally occurring enzymes and bioengineered enzymes or mixtures thereof. Examples of enzyme families include kinases, dehydrogenases, oxidoreductases, GTPases, carboxyl transferases, acyl transferases, decarboxylases, transaminases, racemases, methyl transferases, formyl transferases, and α-ketodecarboxylases.

As used herein, the term “aptamers” means a single-stranded, partially single-stranded, partially double-stranded or double-stranded nucleotide sequence capable of specifically recognizing a selected non-oligonucleotide molecule or group of molecules. In some embodiments, the aptamer recognizes the non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (Pl\ddot{υ}ckthun, The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “antibiotics” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics include, but are not limited to, penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim, and sulfamethoxazole.

The term “therapeutic agents” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

As used herein, the term “hormones” generally refers to naturally or non-naturally occurring hormones, analogues and mimics thereof. In certain embodiments, the term “hormones” refers to any hormones used in therapeutic treatment, e.g., growth hormone treatment. As used herein, “growth hormone” or “GH” refers to growth hormone in native-sequence or in variant form, and from any source, whether natural, synthetic, or recombinant. Examples include human growth hormone (hGH), which is natural or recombinant GH with the human native sequence (somatotropin or somatropin), and recombinant growth hormone (rGH), which refers to any GH or variant produced by means of recombinant DNA technology, including somatrem, somatotropin, and somatropin. In one embodiment, hormones include insulin.

As used herein, the term “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “substantially” means a proportion of at least about 90%, at least about 95%, at least about 98%, at least about 99% or more, or any integer between 90% and 100%. In some embodiments, the term “substantially” can include 100%.

As used herein, the term “comprising” means that other elements can also be present in addition to the defined elements presented. The use of “comprising” indicates inclusion rather than limitation.

The term “consisting of” refers to the components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise explained, all technical and scientific terms used herein have the same meaning as commonly understood by one of ordinary skill in the art to which this disclosure belongs. Definitions of common terms in diseases and disorders, separation and detection techniques can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

This invention is further illustrated by the following example which should not be construed as limiting.

The contents of all references cited throughout this application, examples, as well as the figures and tables are incorporated herein by reference.

### Examples

### Example 1

### Exemplary Materials and Methods for Fabrication and Characterization of Annulus Fibrosus (AF)-Mimic Silk Scaffolds

Although degenerative IVD disease constitutes a large healthcare problem, in surgical treatments for IVD diseases, degenerated AF tissues showed little regeneration, due to their avascular structure (Goins et al., 2005). Thus, tissue engineering of the AF is a potential option for IVD repair. The AF consists of a series of loosely connected concentric layers (lamellae) of highly orientated type I collagen tissue that enclose the nucleus pulpous (NP) (O'Halloran and Pandit, 2007). In tissue engineering aimed at functional tissue restoration, the scaffold plays an important role as a functional template that guides the cellular remodeling process and can potentially provide the cells with temporary protection from unfavorable local implantation environments (Gruber et al., 2004). Various biomaterials have been attempted for AF tissue replacement (Gruber et al., 2006; Sato et al., 2003; Shao and Hunter, 2007). However, these systems have not met the requirements for AF structure and mechanical properties.

Exemplary silk scaffolds utilized herein can be adapted for use in bone and cartilage tissue engineering in vitro and in vivo, due to their impressive mechanical properties, biocompatibility and biodegradability (Hofmann et al., 2006; Kim et al., 2007). At least two types of scaffold morphologies formed from silk were shown in the Examples herein. For example, one mimicked the lamellar features of the native IVD associated with the AF region, and another was a porous spongy scaffold, which was served as a control in Examples 1-4 herein. Toroidal scaffolds were formed from lamellar and porous materials. Thus, morphological features were addressed with scaffold designs to emulate the AF.

Below describes exemplary materials and methods for fabrication and characterization of annulus fibrosus (AF)-mimic silk scaffolds.

Isolation and Culture of Annulus Fibrosus (AF) Cells.

AF cells were isolated from porcine intervertebral disc (IVD). For example, intervertebral discs were obtained from the lumbar disc of pigs (aged 2-3 weeks). The spine was sectioned between each of the lumbar discs from T10 to L5. The muscles and tendons were removed and the column was sectioned transversely in the middle of each disc. The surrounding AF was separated from upper and lower vertebral cartilage and excised so that the surrounding ligament to which it is joined was discarded. Cells from the AF tissues were isolated by 1-2 h digestion at 37° C. in 0.05% pronase (Boehringer-Mannheim), followed by overnight digestion at 37° C. in 0.2% collagenase (Worthington Biochemicals, Lakewood, N.Y., USA), using modified DMEM/F12 (Gibco BRL, Grand Island, N.Y., USA) medium with 5% fetal calf serum (FCS; Gibco BRL), 4.8 mM CaCl₂ and 40 mM HEPES buffer (Sigma-Aldrich). After 18 h of shaking, the completely digested specimens had released cells, which were confirmed by phase-contrast microscopy. The digested samples were centrifuged at 250×g for 5 min to isolate the AF cells for counting. The cells were counted using a haemocytometer and cell numbers and viability were determined using a trypan blue exclusion test. The cells were then plated at a density of 1.5×10⁵ cells/cm² and placed at 37° C. in a 5% CO₂ incubator. The DMEM/F12 culture medium, which included 10% fetal bovine serum (FBS; Gibco), 1% antibiotic-antimycotic (Gibco), 50 μg/ml ascorbic acid (Sigma, St Louis, Mo., USA) was changed every other day. The primary AF cells were passaged twice before the experiments.

Preparation of Silk Solution.

Silk fibroin (SF) solutions were prepared according to procedures described previously, e.g., in Kim et al., (2005a) Biomaterials 26: 4442-4452 and Kim et al., (2005b) Biomaterials 26: 2775-2785. For example, about 6-8% w/v silk fibroin solution was prepared from B. mori silkworm cocoons. The cocoons were extracted in a 0.02 M Na₂CO₃ solution, dissolved in a 9.3 M LiBr solution and subsequently dialyzed against distilled water.

Preparation of Lamellar Silk Scaffolds.

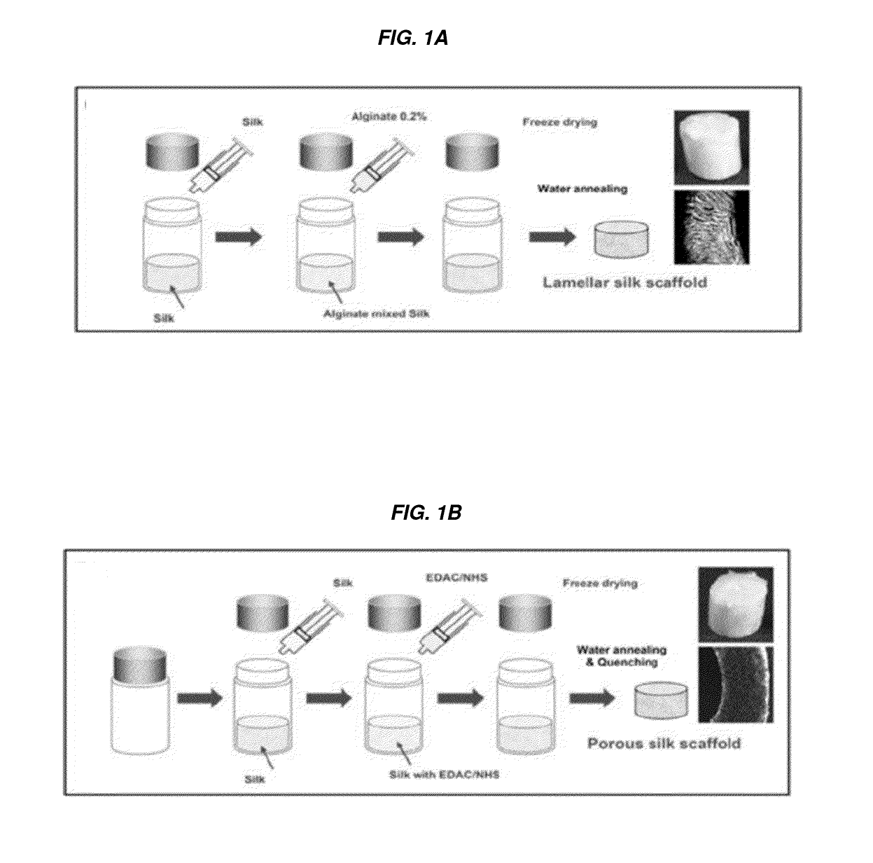

To generate lamellar structure, no salt was added and instead about 1.5 ml 4% SF/0.2% sodium alginate solution mixture was added to a silicone mold (12 mm diameter, 5 ram thick) with one side capped with parafilm. The molds were immediately placed in a freezer at about −80° C. for about 2 h. Subsequently, the scaffolds were lyophilized for about 2 days and then water-annealed for about 6 h to generate the insoluble state of silk by inducing β-sheet crystallinity (Jin et al., 2005). The scaffolds were then submerged in water for about 24 h to remove the mixed alginate. Toroidal disk scaffolds were formed out of the lamellar structure to generate an outer diameter of (o.d.) 8 mm, inner diameter of (i.d.) 3.5 mm and height of 2-3 mm, using disposable punches (Acuderm, Fort Lauderdale, Fla., USA). The toroidal silk disks were submerged in about 70% ethanol (EtOH) for sterile cell cultivation and were conditioned with the culture medium overnight before seeding cells (FIG. 1A).

Preparation of Porous Scaffolds.

Aliquots (2 ml) of 4% silk fibroin solution with about 50 mM carbodiimide (EDAC) and about 20 mM N-hydroxysuccinimide (NHS) in Teflon cylinder containers were kept at room temperature for about 2 h. The containers were then placed in a freezer at about −80° C. for about 2 h. Subsequently, the scaffolds were lyophilized for about 2 days and removed from the containers. To remove the EDAC/NHS residue, the lyophilized silk sponge was suspended in 10 ml quenching solution (5:1 mixture of 0.25 M NaHSO₃ solution and 0.5 N H₂SO₄), and toroidal disk scaffolds were formed out of the porous material using a scalpel with o.d. 8 mm, i.d. 3.5 mm and height 2-3 mm. The silk sponges were submerged in about 70% EtOH for sterilization in preparation for cell cultivation experiments after washing with distilled water for about 1 day. Before cell seeding, the scaffolds were conditioned overnight with the culture medium (FIG. 1B).

In Vitro Cultivation of Silk Scaffolds.

To generate the AF tissues, AF cells were seeded in the toroidal disk scaffolds with lamellar and porous features and then transferred to six-well culture plates and cultured in AF cell culture medium for 2 weeks. The medium consisted of DMEM/F12 (Gibco) supplemented with 10% FBS (Gibco), 1% antibiotic-antimycotic (Gibco) and 50 μg/ml ascorbic acid (Sigma).

Scanning Electron Microscopy (SEM) and Confocal Microscopy.

Cross-sections of the scaffolds prior to and after cell seeding were examined by SEM (Zeiss FESEM Supra55VP, Oberkochen, Germany). The samples were fixed for about 24 h with 0.4% glutaraldehyde and then dehydrated in a series of graded ethanols prior to coating with gold/palladium for 3 min before SEM observation. Autofluorescence images of hydrated silk scaffolds were observed using a Leica TCS SP2 AOBS microscope (Leica, Mannheim, Wetzlar, Germany) with Leica confocal software. Excitation for autofluorescence was at 488 nm and emission was collected at 500-535 nm.

Fourier Transform Infrared Spectroscopy (FTIR).

FTIR analysis was performed using a FTIR 6200 spectrometer (Jasco, Tokyo, Japan), equipped with a deuterated triglycine sulphate detector and a multiple-reflection, horizontal MIRacle ATR attachment (using a Ge crystal). For each measurement, 32 scans were coded with resolution 4/cm, with the wave number in the range 400-4000/cm. Fourier self-deconvolution (FSD) of the infrared spectra covering the amide I region (1595-1705/cm) was performed using Opus 5.0 software. Deconvolution was performed using Lorentzian line shape with a half-bandwidth of 25/cm and a noise reduction factor of 0.3. FSD spectra were curve-fitted to measure the relative areas of the amide I region components.

Cell Viability in 3D Scaffolds.

Cell viability in silk scaffolds was screened using a live/dead kit (Molecular Probes, Eugene, Oreg., USA). Following the manufacturer's instructions, the in vitro sample was treated in a solution for about 40 min. The solution was a mixture of three components: 2 mM ethidium homodimer-1, phosphate-buffered saline (PBS) and 4 mM calcein AM. After washing in sterilized PBS, the sample-embedded slide was observed using a Leica TCS SP2 AOBS microscope with Leica confocal software. The region of interest was selected from z-plane images to include either the surface or the internal pores, beginning with a bottom section at least 1 mm above the surface of the scaffolds. Depth-projection micrographs were obtained from 20 horizontal sections imaged at a depth distance of 50 mm from each other. Live cells were visualized in green and dead cells in red. Viability of the cells was measured by dividing the number of viable cells (green cells) with that of total cells (green+red cells), determined using Image J.

Histological Analysis.

After macroscopic observation, the tissues were fixed with ˜4% formalin for about 24 h. They were then embedded in paraffin and sectioned at 4 μm. Serial sections were stained with haematoxylin and eosin (H&E). Immunohistochemistry was also carried out to screen for expression of type I collagen as the major ECM protein of AF. The sections were washed sequentially in 70% ethanol and PBS and treated with 3% H₂O₂ in PBS, and 0.15% Triton X-100 was added. Once blocked with 1% bovine serum albumin (BSA) solution, they were reacted with a monoclonal antibody raised against porcine type I collagen (1:200; Chemicon, Temecula, Calif., USA) for 1 h, followed by addition of a biotinylated secondary antibody. The protein was then detected using a horseradish peroxidase-conjugated avidin system (Vector Laboratories, Burlingame, Calif., USA). The immunostained sections were counterstained with Mayer's haematoxylin (Sigma) before microscopic examination using a Leica DMIL light microscope (Wetzlar, Germany).

Biochemical Assays for DNA, GAGs and Collagen Content.

The recovered samples (n=4) for DNA and GAGs were digested for 16 h with papain solution (125 μg/ml papain, 5 mM L-cysteine, 100 mM Na₂HPO₄, 5 mM EDTA, pH 6.2) at 60° C. DNA content was measured using the PicoGreen DNA Assay according to the protocol of the manufacturer (Molecular Probes). After centrifugation, a 25 μl aliquot of supernatant was taken from each sample and placed into 96-well plates, with each well containing 75 μl 1× TE buffer. A standard curve was generated using λ phage DNA in 0, 2.5, 5 and 10 μg/ml concentrations. To each well, 100 μl 1:200 dilution of Quant-iT PicoGreen reagent was added and read using a fluorimeter with an excitation wavelength of 480 nm and an emission wavelength of 528 nm. Total GAG content was analyzed using a 1,9-dimethylmethylene blue (DMB) assay (Whitley et al., 1989). Individual samples were mixed with the DMB solution and the absorbance was measured at 525 nm. The total GAG of each sample was extrapolated using a standard plot of shark chondroitin sulphate (Sigma) in the range 0-100 μg/ml.

The tissue-engineered constructs were digested with pepsin solution (1 mg/ml pepsin, pH 3.0) at 4° C. for 48 h to determine total collagen content. Total collagen was measured using the method reported in Park et al. (2005) Artif Organs 29: 838-845. A dye solution, pH 3.5, was prepared with Sirius red dissolved in picric acid saturated solution (1.3%; Sigma) to a final concentration of about 1 mg/ml. The digested samples were dried at 37° C. in 96-well plates for about 24 h and then reacted with the dye solution for about 1 h on a shaker. The samples were then washed five times with 0.01 N HCl and the dye-sample complex in each well was resolved in 0.1 N NaOH and the absorbance read at 550 nm (Versa MAX, Molecular Devices, Sunnyvale, Calif., USA). Total collagen in each sample was extrapolated using a standard plot of bovine collagen (Sigma) in the range 0-500 μg/ml.

Real-Time PCR.

Cultured scaffolds (n=4/group) were transferred into 2 ml plastic tubes and 1.0 ml Trizol was added. The scaffolds were chopped with micro-scissors on ice. The tubes were centrifuged at 12 000×g for 10 min and the supernatant was transferred to a new tube. Chloroform (200 ml) was added to the solution and incubated for 5 min at room temperature. The tubes were again centrifuged at 12 000×g for 15 min and the upper aqueous phase was transferred to a new tube. One volume of 70% ethanol (v/v) was added and applied to an RNeasy Minispin column (Qiagen, linden, Germany). The RNA was washed and eluted according to the manufacturer's protocol. The RNA samples were reverse-transcribed into cDNA, using oligo (dT)-selection according to the manufacturer's protocol (High Capacity cDNA Archive Kit, Applied Biosystems, Foster City, Calif., USA). Collagen type Iα1 (ColIα1) and aggrecan levels were quantified using the Mx3000 Quantitative Real Time PCR system (Stratagene, La Jolla, Calif., USA). All data analysis employed Mx3500 software (Stratagene), based on fluorescence intensity values after normalization with an internal reference dye and baseline correction. Differences in gene expression were evaluated using the comparative Ct method (^{ΔΔ}Ct comparison). Ct values for samples were normalized to an endogenous housekeeping gene. PCR reaction conditions were 2 min at 50° C., 10 min at 95° C., then 50 cycles at 95° C. for 15 s, and 1 min at 60° C. The data were normalized to the expression of the house-keeping gene, glyceraldehyde-3-phosphate-dehydrogenase (GAPDH), within the linear range of amplification and differences (Kim et al., 2005a). The GAPDH probe was labeled at the 5′ end with fluorescent dye VIC and with the quencher dye TAMRA at the 3′ end. Primer sequences for the porcine GAPDH gene were: forward 5′-TCG GAG TGA ACG GAT TTG G-3′ (SEQ ID NO. 1), reverse 5′-CCA GAG TTA AAA GCA GCC CT-3′(SEQ ID NO. 2), probe 5′-ACG CAG TCC TCT CCA GTG GCG AA-3′ (SEQ ID NO. 3). Primer sequences for the porcine collagen type Iα (ColIα1) gene were: forward 5′-AGA AGA AGA CAT CCC ACC AGT CA-3′ (SEQ ID NO. 4), reverse 5′-AGA TCA CGT CAT CGC ACA ACA-3′ (SEQ ID NO. 5), probe 5′-AAC GGC CTC AGG TAC CAT GAC CGA-3′ (SEQ ID NO. 6). Primer sequences for the porcine aggrecan gene were: forward 5′-CCC AAC CAG CGT GAC AAC TT-3′(SEQ ID NO. 7), reverse 5′-CCT TCT CGT GCC AGA TCA TCA-3′ (SEQ ID NO. 8), probe 5′-ACG CAG TCC TCT CCA GTG GCG AA-3′ (SEQ ID NO. 9). Probes were purchased from Assay on Demand (Applied Biosciences).

Mechanical Strength.

Tensile tests were performed on an Instron 3366 testing frame (Grove City, Pa., USA) equipped with a 10 N capacity load cell and Biopuls™ pneumatic clamps. The selected toroidal disk scaffolds (height 2 mm) were hydrated in 0.1 M PBS for >30 min, anchored to custom submersible ring-testing grips and submerged in a temperature-controlled Bioplus bath (37±0.3° C.) filled with PBS for at least 5 min prior to testing. A displacement control mode was used, with a crosshead displacement rate of 1 mm/min. Taking the initial gauge length as the non-deformed average diameter (Dₒ+Dₘ/2), the original cross-sectional area (4.5 mm²) and the effective material cross-sectional area (9.0 mm²), determined based on an assumed uniform and constant wall thickness (WT=2.25 mm) and measured sample heights (T=2 mm), the tensile stress and strain were graphed and the initial ‘linear elastic modulus’, elongation to failure and ultimate tensile strength determined. The initial ‘linear elastic modulus’ was calculated by using a least-squares' (LS) fitting between 0.1 N load (porous scaffolds) or 0.01 N load (lamellar scaffolds) and 10% strain past that point. Ultimate tensile strength (UTS) was determined as the highest stress value attained during the test. The elongation to failure was determined as the last data point before a>10% decrease in load (failure strain minus the strain corresponding to 0.1 N load noted earlier). The toroidal disk scaffolds were stretched through an elliptical shape and eventually fully stretched to a linear shape.

Statistical Analysis.

Statistical differences in biochemical and mechanical quantitative analysis were determined using a Mann-Whitney U-test (independent t-test, SPSS). Statistical significance was assigned as *p<0.05, **p<0.01 and ***p<0.001, respectively.

### Example 2

### Physical Features of Exemplary AF-Mimic Silk Scaffolds

The success of a cell-based, polymeric tissue-engineered disc graft relies, in part, on its ability to function as a three-dimensional (3D) support matrix, mimicking the native structure, in order to help with graft integration and to support cell proliferation and production of tissue-specific ECM (Temenoff and Mikos, 2000). In this Example, a simplified freeze-drying technique was utilized to generate lamellar scaffolds for engineering functional units of the AF. This lamellar scaffold was compared to non-lamellar porous scaffolds as controls for AF-like tissue formation. In this Example, it was sought to determine whether a lamellar scaffold system based on silk can provide improved AF tissue formation and function in vitro. To evaluate the utility of this scaffold for tissue engineering, cell growth was characterized by scanning electron microscopy (SEM), histology and immunostaining. In addition, chemical analysis (collagen and glycosaminoglycans), qPCR and mechanical testing were used to assess outcomes.

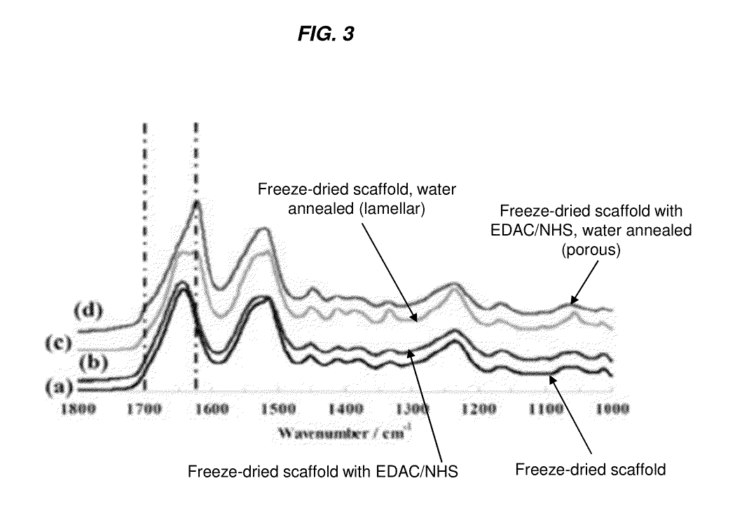

The scaffolds prepared by about −80° C. freezing in the presence of sodium alginate solution (as described in Example 1) possessed lamellar features similar to the AF region of the native IVD (FIG. 2A). The other group of porous spongy scaffolds (prepared in the presence of EDAC and NHS) were served as a control (FIG. 2B). Observations of silk scaffolds by SEM and confocal microscopy (hydration) revealed that the interlamellar distance in the lamellar scaffolds was about 150 μm to about 250 μm, and the average pore size of the porous scaffolds was about 100 μm to about 250 μm. FTIR analysis of the silk scaffolds showed characteristic peaks for silk II (the fl-sheet crystalline state) at 1701/cm and 1623/cm (amide I) (Kim et al., 2005b). Lamellar silk scaffold structures were achieved by freeze-drying and water-annealing. EDAC/NHS-doped silk gel was freeze-dried with water annealing to generate the porous scaffolds. As shown in Table 1 below and FIG. 3, both scaffolds showed high fl-sheet content after water annealing. The crystallinity of the lamellar and porous scaffolds were about 38% and about 46%, respectively. EDAC/NHS led to a slight increase in crystallinity (˜8%) of the porous scaffolds. However, EDAC/NHS did not induce β-sheet formation before water annealing.

TABLE 1
Beta sheet content in various embodiments of freeze-dried silk scaffolds

| Freeze-dried silk scaffolds | β-sheet/% |
|---|---|
| Freeze-dried | 17% |
| Freeze-dried w/EDAC/NHS | 17% |
| Freeze-dried - water-annealed | 38% |
| Freeze-dried w/EDAC/NHS - water-annealed | 46% |

To generate the lamellar structure of AF tissue, alginate fiber was previously reported to be used in combination with a rotating spinner flask (Shao and Hunter, 2007), which does not involve any freeze-drying technique. In contrast, in some embodiments described herein, the fabrication of a lamellar structure for AF tissue was accomplished using a freeze-drying technique (e.g., bottle freeze-drying technique). In some embodiments, lamellar shapes from pure silk solution (e.g., with no additives) were generated by a freeze-drying technique with an inter-lamellar distance (10-20 μm) too narrow for adequate cell seeding and function. To generate larger inter-lamellar distances, in some embodiments, 0.2% sodium alginate was mixed into silk solution, which led to 150-250 μm inter-lamellar distances, due to different ice crystal sizes during freezing (FIGS. 2A-2B). Based on a scaffold preparation method used in Examples 1 and 2, the lamellar structures shown herein were not circumferentially orientated. Circumferentially orientated structures can be important for the functional restoration of AF tissue. Thus, in some embodiments, the lamellar structures described herein can be produced to be circumferentially orientated.

In order to fabricate porous silk scaffolds, in some embodiments, a salt-leaching approach as previously described can be used. However, salt-leaching was less preferably used to produce small porous (<250 μm) shapes. As an alternative technique, EDAC/NHS was added into the silk solution. As a result, EDAC/NHS-mixed silk solution formed a gel after 2 h, due to the ability of EDAC/NHS to induce crosslinking between amine functional groups on the amino acids in the silk protein chains. The added EDAC/NHS resulted in only a small increase in crystallinity after water annealing and produced uniform 100-250 μm pores (FIG. 3).

### Example 3

### Morphologies and Responses of Cells Seeded in Exemplary AF-Mimic Silk Scaffolds

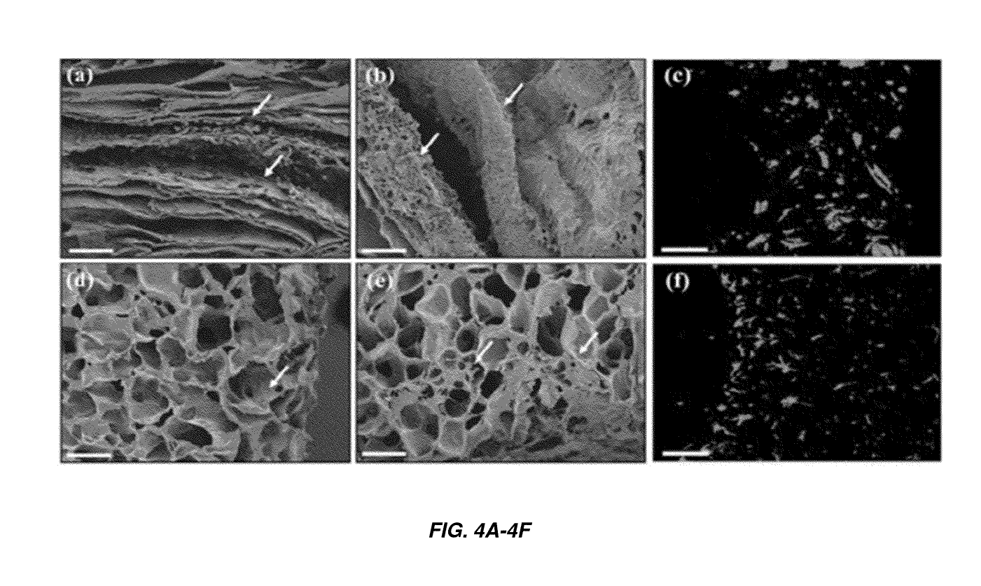

To generate the AF tissues, AF cells were seeded in the lamellar silk scaffolds and porous silk scaffolds as described in Examples 1 and 2, and then cultured in AF cell culture medium for 2 weeks. The seeded porcine AF cells were supported in the lamellar silk scaffolds (described in Examples 1 and 2) over 2 weeks, based on analysis of the confocal images. The proliferating cells spread along the lamellar walls (FIGS. 4A-4B), while cells on the porous scaffold (as described in Examples 1 and 2) could not penetrate into the interior from the surface of the scaffold (FIGS. 4D-4E). Most of the attached and proliferated cells (>90%) survived in both the scaffolds, as shown by live/dead staining (FIGS. 4C and 4F).

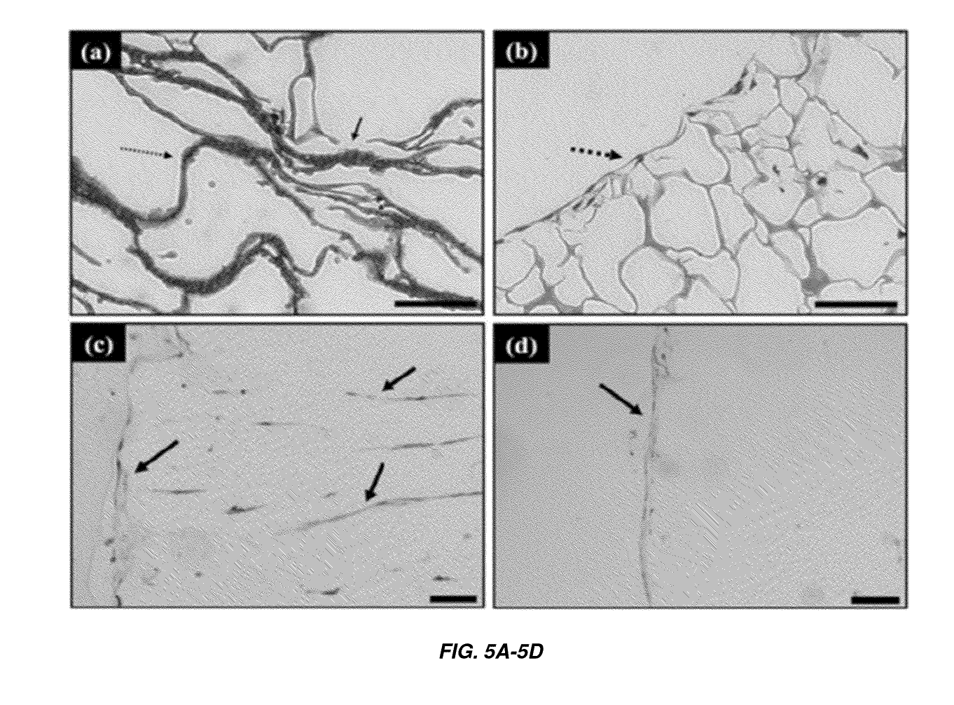

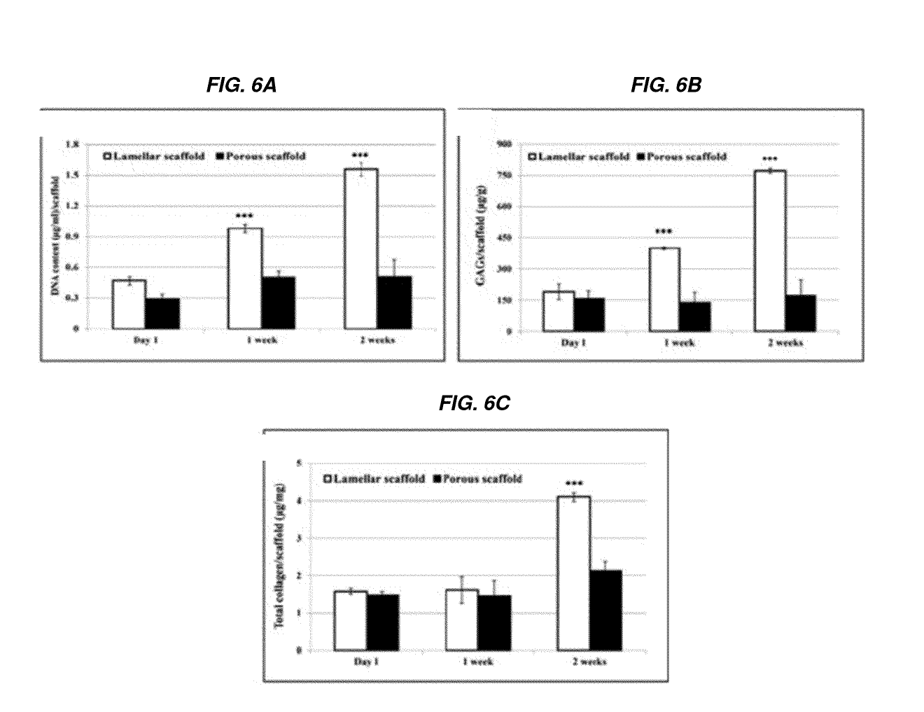

For the identification of cell distributions and AF-specific ECM molecules, thin sections of each specimen were stained with H&E and for type I collagen. Cells seeded in the lamellar silk scaffolds showed homogeneous distribution and proliferation following the lamellar walls after 2 weeks (FIG. 5A). In contrast, cells seeded in the porous silk scaffolds appeared to spread more prominently along the surfaces of the scaffolds (FIG. 5B). From type I collagen staining, the cells synthesized ECM for AF tissue in both types of scaffolds. In particular, type I collagen staining was homogeneously distributed within the entire lamellar structure (FIG. 5C), while the porous scaffolds showed more staining at the scaffold surface (FIG. 5D).

DNA, GAGs and total collagen content in the lamellar scaffolds were significantly higher than in the porous scaffolds. The DNA content in the porous silk scaffold increased slightly over time, while in the lamellar scaffolds the increase was significant at 1 and 2 weeks (FIG. 6A). In addition, the lamellar silk scaffolds were determined to have increasing GAGs and collagen throughout the culture period. The GAG content was about five times higher after 2 weeks than the porous silk scaffolds. The amount of GAGs (μg/g wet weight) in the lamellar and porous silk scaffolds at 2 weeks were 771.2±10.8 and 173.3±74.3, respectively (p<0.001) (FIG. 6B). The total collagen content (4.1±0.1 μg/mg wet weight) of the lamellar silk scaffolds was two-fold higher than the value in the porous scaffolds (2.1±0.3 μg/mg) (FIG. 6C).

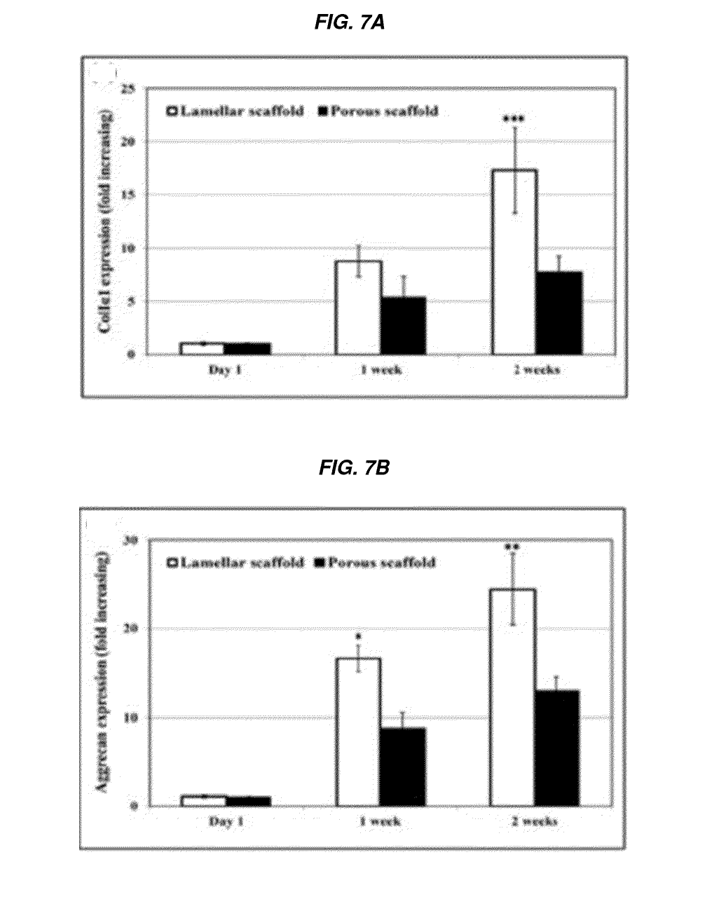

To further support the results from histological observation, transcript levels related to AF differentiation markers ColIα1 and aggrecan were analyzed. The mRNA levels of ColIα1 and aggrecan were significantly increased in the lamellar silk scaffold with the time in culture. In contrast, mRNA levels of these genes did not significantly increase in the porous silk scaffolds, while they were around two times higher in the lamellar silk scaffolds after 2 weeks (FIGS. 7A-7B).

As noted above, the lamellar shaped scaffolds supported cell seeding and proliferation to form an AF-like tissue. SEM images showed even distribution of cells in the lamellar scaffolds. After 2 weeks, entire areas of the walls were covered by proliferating cells. Thus, the cells were able to penetrate into the inside of the scaffold. In contrast, porous silk scaffolds showed proliferating cells mainly on the surfaces of the scaffolds (FIGS. 4A-4F). Low cell penetration on the porous silk scaffold was previously reported where about 150-250 μm pores in silk scaffolds showed non-uniform distribution of tissue growth throughout the structure (Chang et al., 2007). Large-pore scaffolds (˜600 μm) and spinner flask dynamic culture were also used to overcome the low cell penetration into the porous silk scaffold (Chang et al., 2010). In another previous report, a collagen honeycomb-shaped scaffold was used to retain cells for better tissue formation (Sato et al., 2003). However, these porous scaffolds did not mimic the AF structure. In contrast, some embodiments of the implantable intervertebral disc devices described herein comprise an AF-mimic scaffold that (a) simulates the lamellar structure of the native tissue; and (b) supports cell penetration into the scaffold interior. For example, seeded AF cells were able to proliferate between the lamella walls in the scaffold (FIGS. 5A-5D).

A functional scaffold generally provides physical support for cell attachment and promotes cell proliferation and desired ECM deposition (Shao and Hunter, 2007), as the physiological properties of the disc are linked to the composition of its ECM (O'Halloran and Pandit, 2007). The major components of the AF ECM are fibrillar collagens and proteoglycans (PGs) (Kluba et al., 2005). The largest and most important PG in the disc matrix includes aggrecan, which consists of a protein core with attached glycosaminoglycans (negatively charged chondroitin sulphate and keratin sulphate) (Ghosh et al., 1980). The chondroitin sulphate molecule generally plays a crucial role in retaining water, which in turn gives the disc its resilient compressive strength (Meisel et al., 2007). When AF cells were seeded onto the lamellar or porous silk scaffolds, AF cells adhered to both scaffolds and synthesized collagen and proteoglycans. The lamellar scaffolds supported more AF tissue-specific features, based on histological, biochemical and gene expression data for collagen and aggrecan (FIGS. 6A-6C; and FIGS. 7A-7B).

### Example 4

### Mechanical Strength of Exemplary AF-Mimic Silk Scaffolds

AF tissue should be mechanically stable, as IVDs are particularly vulnerable to fatigue failure, especially when the bending moment is high (Aubin et al., 2004). Failure of the AF or damage to the collagenous network is a potential cause of disc herniation. With disc degeneration, failure stresses were significantly reduced (Acaroglu et al., 1995). This mechanical property of human AF tissue is reported at 0.9-3.8 MPa to failure strength, 20-60% strain and 0.4-0.5 MPa tensile modulus (Fujita et al., 1997; Green et al., 1993; Iatridis et al., 2005).

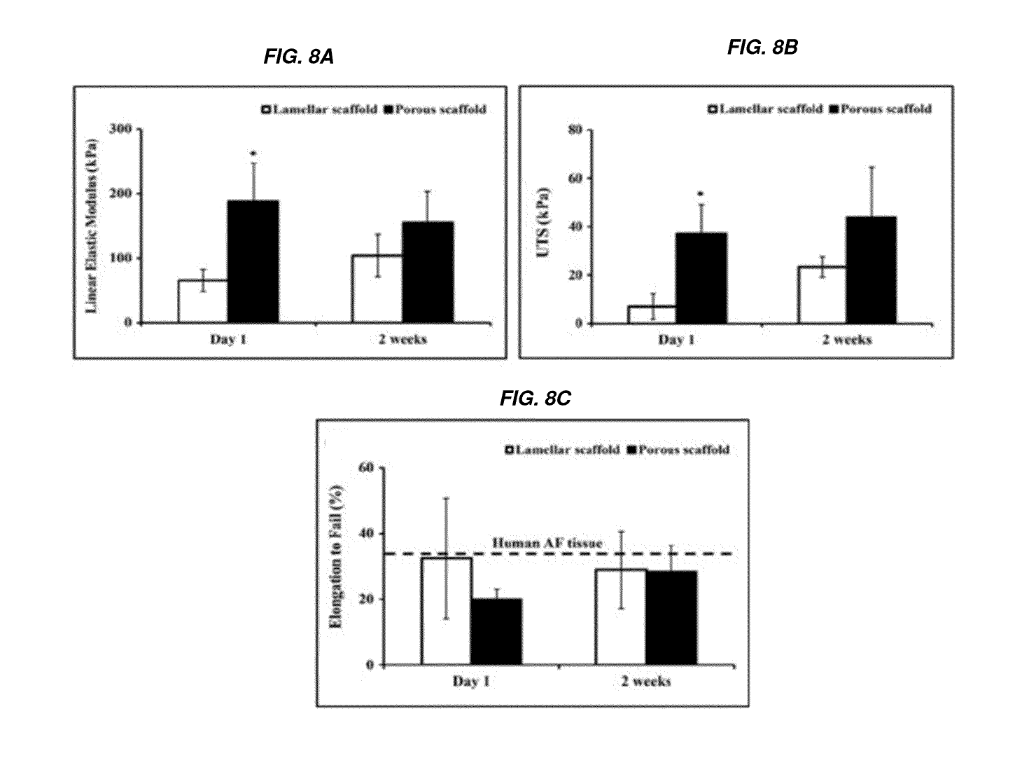

Accordingly, cell-seeded scaffolds from cell culture (Example 3) were evaluated for their mechanical properties. The porous scaffolds had a higher ‘linear elastic modulus’ and ultimate tensile strength at day 1, while both the lamellar and porous scaffolds showed similar values after 2 weeks in culture (FIGS. 8A-8B). There were no significant differences in elongation to failure between the lamellar and porous scaffolds (FIG. 8C).

As noted above, silk scaffolds were assessed in the elongation-to-failure mode in this Example and the lamellar and porous scaffolds showed similar properties to that of the native tissue. In measuring linear elastic modulus and ultimate tensile strength (UTS), lamellar scaffolds were determined to have weaker properties than porous scaffolds in the earlier time points, but the values did not show any statistical difference after 2 weeks (FIGS. 8A-8B). Without wishing to be bound by theory, although the characterization method used in this Example measured the mechanical properties while maintaining the original structure, a highly complex non-linear test of the structural stiffness can be performed when the deformation of the toroid scaffold itself is considered. However, the force used for toroid scaffold deformation was negligible (<0.1 N for porous and 0.01 N lamellar scaffolds) under PBS hydrated measuring conditions, compared to the force needed for stretching the scaffolds.

While the lamellar silk scaffolds shown in Examples 1-4 were not circumferentially orientated, the lamellar shape architecturally resembled native AF. Accordingly, presented herein, in part, is a method to fabricate lamellar structure using silk material and to provide dimensions sufficient for cell proliferation and production of ECM. In addition, histological and biochemical analyses, immunohistochemistry and gene expression profiling revealed time-dependent development of the AF phenotype from the seeded cells. The cells within the lamellar scaffolds maintained the architecture of a native AF over at least a period of 2 weeks of culture. Thus, a biphasic tissue-engineered IVD tissue mimetic comprising a lamellar silk scaffold to provide structure and function of an AF can be produced.

### Example 5

### An Exemplary Method to Fabricate Native-Like Annulus Fibrosus (AF) Region of an Intervertebral Disc

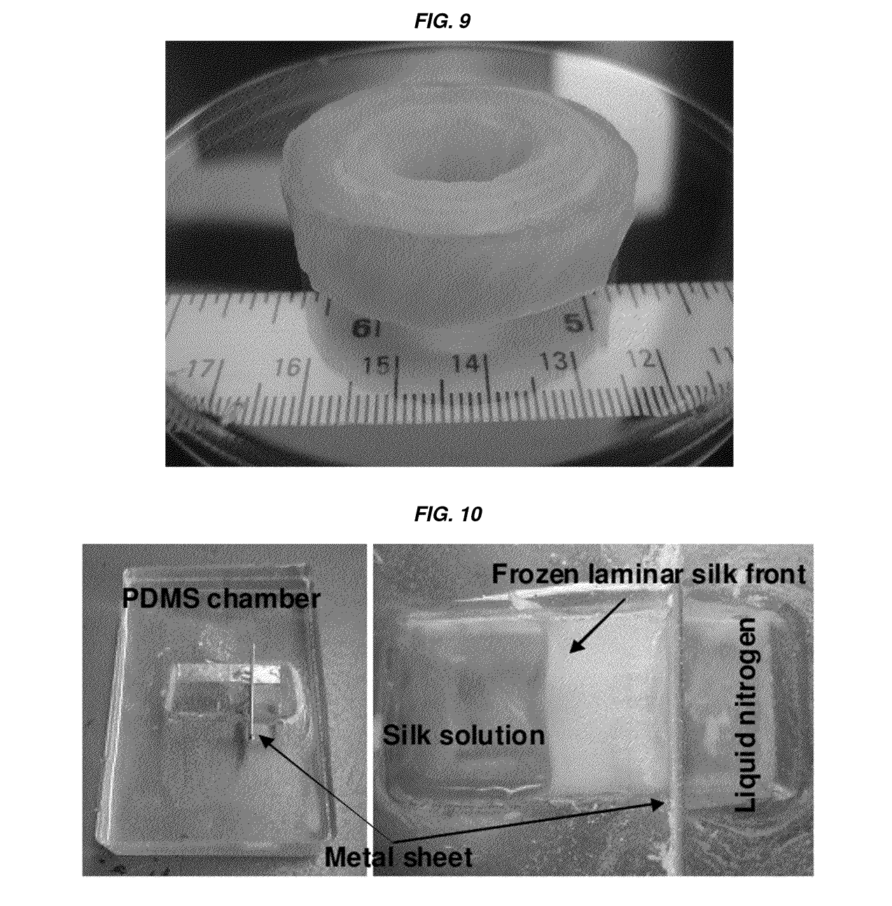

Described herein is an exemplary method to fabricate a full sized annulus fibrosus (AF) region of an intervertebral disc mimicking its native like cross alignment (see, e.g., FIG. 9). For example, the method can be used to mimic native like 30 degree cross-aligned, multiple layered structure found in native annulus fibrosus (AF) region of an intervertebral disc. The method of fabricating aligned scaffolds described in this Example included directional and controlled cooling. These fabricated aligned scaffolds were then cut in specific directions to achieve about 30 degree cross alignment similar to native AF region. Further, the entire AF region was created by wrapping the aligned scaffold strips to mimic circular orientation of the AF region. The developed model is not only simple to fabricate but also requires minimal resources to make; thus it is highly cost effective. Accordingly, the developed intervertebral disc model, particularly a tissue-engineered AF scaffold, can serve as a scaffolding material for cell and matrix alignment and as an intervertebral disc graft.

Exemplary materials and methods used for fabrication of a native-like AF silk-based tissue device with a 30 degree cross aligned structure are described below:

Degummed silk fibers from cocoons were used to prepare a silk solution. An exemplary protocol for preparation of degummed silk fibers from cocoons is described below (any modifications readily known to a skilled artisan, e.g., different concentrations and/or types of carbonate salts, or boiling time for degumming process, are encompassed by the protocol):
1. Cut dried cocoons (e.g., with scissors) into pieces (e.g., 4 pieces);
2. Heat a pre-determined volume of water until boiling (e.g., prepare 2 separate containers such as glass beakers filled with water (e.g., 3 L water) each and heat it up until boiling);
3. Depending on the volume of water from step (2), weigh an amount of sodium carbonate sufficient to prepare a 0.02 M sodium carbonate solution;
4. Add sodium carbonate to the container with water when water starts to boil and let it dissolve.
5. Put the cut cocoon pieces in the boiling water with 0.02 M sodium carbonate, and stir.
6. Boil for at least about 10 minutes with occasional stiffing;
7. After boiling the cocoon pieces in water for at least about 10 minutes, transfer the silk fibers to a separate container with boiling water containing 0.02 M sodium carbonate.
8. Boil for at least another 10 min with occasional stirring;
9. Take the degummed fibers out of the boiling water and rinse with cold water (e.g., 5-7 washes) until most or all sodium carbonate is removed;
10. Optionally remove excess water; and
11. Air dry the degummed fibers for at least about 12 hours.

After degummed silk fibers are prepared, a silk solution can be prepared by an exemplary protocol described below (any modifications readily known to a skilled artisan, e.g., different concentrations and/or types of salts, incubation time and/or temperatures, are encompassed by the protocol):
1. Prepare a solution of Lithium Bromide (LiBr), e.g., at a concentration of about 9.3M in a container (e.g., a glass beaker);
2. Incubate the LiBr solution at about 60° C. for about 10 mins (e.g., cover the LiBr solution with aluminum foil and keep the beaker in the oven at about 60° C. for 10 min);
3. Add about 10 grams of degummed silk fibers to the LiBr solution. Gentle mixing can help faster dissolution of the degummed silk fibers in the LiBr solution;
4. Keep the fibers with the LiBr solution in the oven for about 1 hr and allow it to completely dissolve. A clear solution is produced when the fibers are completely dissolved;
5. After silk fibers dissolve, perform dialysis to remove LiBr;
   a. Fill a container with distilled water;
   b. Pour the silk solution using a syringe into a dialysis cassette (12 kDa) and immerse them into the container with distilled water for dialysis; and
   c. Change water with fresh distilled water every about 1 hr for the next 4 hrs. Then change water every about 6-8 hrs for another 3-4 times.
6. After dialysis, transfer the silk solution from the dialysis cassette into a clean container (e.g., a glass beaker).
7. Centrifuge at about 5000 rpm for about 5 mins;
8. Optionally aliquot the silk solution and store at 4° C. until use;
9. If desirable, determine weight percentage of silk in a solution, e.g., by drying (e.g., in oven) about 1 ml of silk solution (e.g., in a small teflon dish) to determine the dry weight of the silk.

To fabricate aligned silk scaffolds with laminar channels (pores), controlled directional freezing method was performed using liquid nitrogen (e.g., at a temperature of about 196° C.). In some embodiments, a custom chamber to perform directional freezing was fabricated using polydimethylsiloxane (PDMS) (FIGS. 10-11). PDMS belongs to a group of polymeric organo silicon compounds that are commonly referred to as silicones. PDMS was selected as a chamber material because of its relative non-conductivity. When liquid nitrogen is poured inside the chamber, using a chamber material that is relatively heat-conductive would not be able to confer directionality to the formed pores. Thus, metal is unlikely to be used to form the entire chamber as it will cool down quickly everywhere when it is in contact with liquid nitrogen, resulting in formation of random pores due to silk freezing in all directions.

As shown in FIGS. 10-11, an exemplary chamber was consisted of a shallow chamber casted out of PDMS (following general method of PDMS casting by mixing the base polymer with the curing agent in the ratio of about 9:1), and the PDMS chamber was divided into smaller chambers using a metal sheet. The metal sheet cools when liquid nitrogen is poured into one chamber and which in turn cools the liquid silk on the other side of the chamber, thus initiating the process of laminar, directional pore formation. Any metal sheets that are relatively good heat conductor can be used. For example, a metal sheet can include zinc, copper, aluminum, iron, or any combinations thereof. In one embodiment, the metal sheet can include a zinc composite metal.

The size of the chamber can be altered depending on the required size of laminar scaffolds. For example, the larger the size of a laminar scaffold to be fabricated, the larger the size of the chamber is needed. A smaller laminar scaffold can be fabricated by using a smaller chamber, or by reducing a larger laminar scaffold into a smaller one. In some embodiments, depending on the chamber material, e.g., if PDMS is used, the width of a laminar scaffold (in the direction of freezing) can be no more than 3-4 cm. This is because, without wishing to be bound by theory, as the freezing front of the silk solution travels down the chamber, e.g., as shown in FIG. 10, the temperature gradient decreases and thus becomes less effective in creating laminar channels beyond 3-4 cm. However, the length and height of the scaffold to be fabricated can be varied in accordance with different needs.

An exemplary protocol for fabrication of a laminar silk scaffold using a silk solution in a custom chamber (e.g., as shown in FIGS. 10-11) is described below (any modifications readily known to a skilled artisan, e.g., different low-temperature liquids, and/or different methods to increase beta sheet crystallinity, are encompassed by the protocol):
1. Pour a silk solution (e.g., ˜5 wt % silk solution) into a first chamber of the PDMS mold;
2. Pour liquid nitrogen into a second chamber that is separated from the first chamber containing the silk solution by a metal sheet, where liquid nitrogen cools the metal sheet which in turn cools the silk solution leading to directional pore formation (e.g., narrow channels). Silk freezing was observed in the first chamber and appeared as laminar channels with a leading front end.
5. Maintain the second chamber at a liquid-nitrogen temperature (e.g., by replenishing the second chamber with liquid nitrogen till a desirable amount (e.g., most or all) of the silk solution freezes in the first chamber
6. Once the desirable amount (e.g., most or all) of the silk solution is frozen, dry or lyophilize the frozen silk. The time of drying, e.g., in a lyophilizer, can depend on the amount/volume of frozen silk.
7. After the frozen silk has been lyophilized till fully dried, immerse the dried silk scaffold in an alcohol solution (e.g., 80% methanol) over a period of time, e.g., overnight. Optionally, one can store the dried silk scaffold in an alcohol solution (e.g., 70% methanol solution) until use or can readily use it for applications.
8. Optionally remove the top and bottom surface or “skin” of the dried silk scaffold, e.g., by removing a thin section using sharp surgical blades, in order to open up any pores which might be closed by the top and bottom silk surface or “skin”.
9. Section the dried laminar (aligned) silk scaffold into a desired size.

The silk solution concentration can vary depending on various applications or users' preference. In some embodiments, it is less desirable to use a silk solution of less than 3 wt %, because, without wishing to be bound by theory, the fabricated silk scaffolds could collapse after drying. Higher silk solution concentration can be used when higher mechanical properties are desired. However, without wishing to be bound by theory, smaller pores can be formed when higher silk solution concentrations are used. FIG. 12A is a SEM image showing an aligned silk scaffold fabricated by a directional freezing approach as described herein, and FIG. 12B shows alignment of fluorescent protein-tagged fibroblast cells grown in the aligned silk scaffold.

After fabrication of a laminar (aligned) silk scaffold, fabrication of a native-like annulus fibrosus with 30 degree cross aligned structure can be performed following an exemplary protocol below:

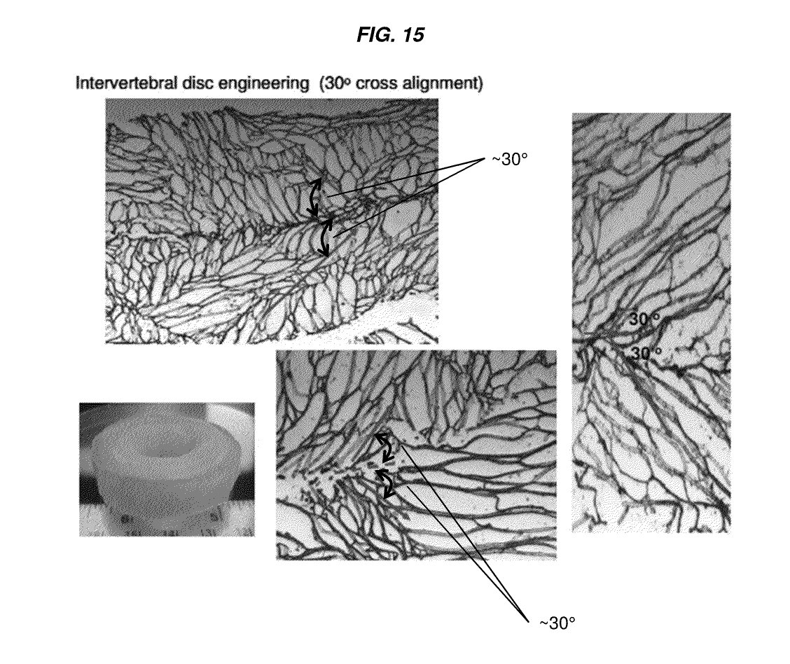

1. If a larger laminar silk scaffold is produced, cut a plurality of thin strips at an angle of about 30 degrees, e.g., using surgical blades or by automated cutting (FIG. 13, top). It is desirable to have the thickness of a strip close to that of a native AF structure, e.g., about 500 μm.
2. Wrap each strip circularly over one another in an alternating direction to mimic the AF region architecture as shown in the bottom left of FIG. 13 such that a 30 degree cross alignment is created as shown in FIGS. 14-15.
3. Repeat step 2 until a desirable number of layers, e.g., 10 or more layers, is reached, e.g., to mimic the size of a native AF region of an intervertebral disc. Native AF can have about 16-20 rings depending on its diameter. FIG. 14 shows an exemplary full-sized annulus fibrosus (AF) region of an intervertebral disc, which was fabricated using the protocol described herein to form a cross-aligned structure as observed in a native AF region.

### REFERENCES

Acaroglu E R, Iatridis J C, Setton L A, et al. 1995; Degeneration and aging affect the tensile behavior of human lumbar annulus fibrosus. Spine 20: 2690-2701.
Anderson J A. 1986; Epidemiological aspects of back pain. J Soc Occup Med 36: 90-94.
Aubin C E, Goussev V, Petit Y. 2004; Biomechanical modelling of segmental instrumentation for surgical correction of 3D spinal deformities using Euler-Bernoulli thin-beam elastic deformation equations. Med Biol Eng Comput 42: 216-221.
Chang G, Kim H J, Kaplan D, et al. 2007; Porous silk scaffolds can be used for tissue engineering annulus fibrosus. Eur Spine J 16: 1848-1857.
Chang G, Kim H J, Vunjak-Novakovic G, et al. 2010; Enhancing annulus fibrosus tissue formation in porous silk scaffolds. J Biomed Mater Res A 92: 43-51.
Feng H, Danfelter M, Stromqvist B, et al. 2006; Extracellular matrix in disc degeneration. J Bone Joint Surg Am 88: 25-29.
Frenkel S R, Di Cesare P E. 2004; Scaffolds for articular cartilage repair. Ann Biomed Eng 32: 26-34.
Fujita Y, Duncan N A, Lotz J C. 1997; Radial tensile properties of the lumbar annulus fibrosus are site- and degeneration-dependent. J Orthop Res 15: 814-819.
Ghosh P, Bushell G R, Taylor T K, et al. 1980; Distribution of glycosaminoglycans across the normal and the scoliotic disc. Spine 5: 310-317.
Goins M L, Wimberley D W, Yuan P S, et al. 2005; Nucleus pulposus replacement: an emerging technology. Spine J 5: 317-324S.
Green T P, Adams M A, Dolan P. 1993; Tensile properties of the annulus fibrosus. Eur Spine J 2: 209-214.
Gruber H E, Hoelscher G L, Leslie K, et al. 2006; Three-dimensional culture of human disc cells within agarose or a collagen sponge: assessment of proteoglycan production. Biomaterials 27: 371-376.
Gruber H E, Leslie K, Ingram J, et al. 2004; Cell-based tissue engineering for the intervertebral disc: in vitro studies of human disc cell gene expression and matrix production within selected cell carriers. Spine J 4: 44-55.
Hegewald A A, Ringe J, Sittinger M, et al. 2008; Regenerative treatment strategies in spinal surgery. Front Biosci 13: 1507-1525.
Hofmann S, Knecht S, Langer R, et al. 2006; Cartilage-like tissue engineering using silk scaffolds and mesenchymal stem cells. Tissue Eng 12: 2729-2738.
Iatridis J C, MaClean J J, Ryan D A. 2005; Mechanical damage to the intervertebral disc annulus fibrosus subjected to tensile loading. J Biomech 38: 557-565.
Jin H J, Park J, Karageorgiou V, et al. 2005; Water-stable silk films with reduced β-sheet content. Adv Funct Mater 15: 1241-1247.
Kim H J, Kim U J, Leisk G G, et al. 2007; Bone regeneration on macroporous aqueous-derived silk 3D scaffolds. Macromol Biosci 7: 643-655.
Kim H J, Kim U J, Vunjak-Novakovic G, et al. 2005a; Influence of macroporous protein scaffolds on bone tissue engineering from bone marrow stem cells. Biomaterials 26: 4442-4452.
Kim U J, Park J, Kim H J, et al. 2005b; Three-dimensional aqueous-derived biomaterial scaffolds from silk fibroin. Biomaterials 26: 2775-2785.
Kluba T, Niemeyer T, Gaissmaier C, et al. 2005; Human annulus fibrosis and nucleus pulposus cells of the intervertebral disc: effect of degeneration and culture system on cell phenotype. Spine 30: 2743-2748.
Marchand F, Ahmed A M. 1990; Investigation of the laminate structure of lumbar disc annulus fibrosus. Spine 15: 402-410.
Meisel R I, Siodla V, Ganey T, et al. 2007; Clinical experience in cell-based therapeutics: disc chondrocyte transplantation. A treatment for degenerated or damaged intervertebral disc. Biomol Eng 24: 5-21.
Mizuno H, Roy A K, Vacanti C A, et al. 2004; Tissue-engineered composites of annulus fibrosus and nucleus pulposus for intervertebral disc replacement. Spine 29: 1290-1297.
O'Halloran D M, Pandit A S. 2007; Tissue-engineering approach to regenerating the intervertebral disc. Tissue Eng 13: 1927-1954.
Park S H, Park S R, Chung S I, et al. 2005; Tissue-engineered cartilage using fibrin/hyaluronan composite gel and its in vivo implantation. Artif Organs 29: 838-845.
Raghunath J, Rollo J, Sales K M, et al. 2007; Biomaterials and scaffold design: key to tissue-engineering cartilage. Biotechnol Appl Biochem 46: 73-84.
Richardson S M, Walker R V, Parker S, et al. 2006; Intervertebral disc cell-mediated mesenchymal stem cell differentiation. Stem Cells 24: 707-716.
Rong Y, Sugumaran G, Silbert J E, et al. 2002; Proteoglycans synthesized by canine intervertebral disc cells grown in a type I collagen-glycosaminoglycan matrix. Tissue Eng 8: 1037-1047.
Sato M, Asazuma T, Ishihara M, et al. 2003; An atelocollagen honeycomb-shaped scaffold with a membrane seal (ACHMS-scaffold) for the culture of annulus fibrosus cells from an intervertebral disc. J Biomed Mater Res A 64: 248-256.
Shao X, Hunter C J. 2007; Developing an alginate/chitosan hybrid fiber scaffold for annulus fibrosus cells. J Biomed Mater Res A 82: 701-710.
Temenoff J S, Mikos A G. 2000; Review: tissue engineering for regeneration of articular cartilage. Biomaterials 21: 431-440.
Venugopal J, Ramakrishna S. 2005; Biocompatible nanofiber matrices for the engineering of a dermal substitute for skin regeneration. Tissue Eng 11: 847-854.
Vepari C, Kaplan D L. 2007; Silk as a biomaterial. Prog Pulpit Sci 32: 991-1007.
Wan Y, Feng G, Shen F H, et al. 2008; Biphasic scaffold for annulus fibrosus tissue regeneration. Biomaterials 29: 643-652.
Whitley C B, Ridnour M D, Draper K A, et al. 1989; Diagnostic test for mucopolysaccharidosis. I. Direct method for quantifying excessive urinary glycosaminoglycan excretion. Clin Chem 35: 374-379.

All patents and other publications identified throughout the specification are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

What is claimed is:

1. An implantable intervertebral disc device comprising:
a silk-based toroidal disc scaffold, the toroidal disc scaffold comprising on its circumferential surface at least two concentric layers of laminar silk scaffold strips;
wherein a first laminar silk scaffold strip comprises at least about 5% of its first porous structures substantially aligned at a predetermined angle to the bottom edge of the first laminar scaffold strip;
wherein a second laminar silk scaffold strip circumferentially wraps around the first laminar silk scaffold strip in a manner such that the second laminar silk scaffold strip comprises at least about 5% of its second porous structures substantially aligned at an angle of about 20° to about 160° with respect to the aligned first porous structures.

2. The device of claim 1, wherein at least about 10%, at least about 20%, at least about 30%, at least about 40%, at least about 50%, at least about 75%, or at least about 90% of the first porous structures are substantially aligned at the predetermined angle to the bottom edge of the first laminar scaffold strip.

3. The device of claim 1, wherein at least about 10%, at least about 20%, at least about 30%, at least about 40%, at least about 50% , at least about 75%, or at least about 90% of the second porous structures are substantially aligned at the angle of about 20° to about 160° with respect to the aligned first porous structures.

4. The device of claim 1, wherein said at least two concentric layers includes a range of about 2 to about 30 concentric layers, or about 10 to about 20 concentric layers.

5. The device of claim 4, wherein at least about 5% of the porous structures between any two successive concentric layers are substantially oriented or aligned at an angle of about 80° to about 140° with respect to each other.

6. The device of claim 1, wherein the predetermined angle ranges from about 10° to about 80°, or about 15° to about 60°, or about 20° to about 40°, or is about 30°.

7. The device of claim 1, wherein the porous structures have a pore size of about 10 μm to about 500 μm, or about 100 μm to about 250 μm.

8. The device of claim 1, wherein an inter-lamellar spacing in the lamellar silk scaffold strips ranges from about 10 μm to about 500 μm, or about 150 μm to about 250 μm.

9. The device of claim 1, further comprising a biocompatible gel surrounded by the silk-based toroidal disc scaffold.

10. The device of claim 9, wherein the biocompatible gel comprises silk fibroin.

11. The device of claim 1, further comprising at least one intervertebral disc cell present in the silk-based toroidal disc scaffold, the biocompatible gel, or both.

12. A method of producing an implantable intervertebral disc device comprising:
a. providing a first and a second laminar silk scaffold strips, wherein the first and the second laminar silk scaffold strips each comprises at least about 5% of its porous structures substantially aligned at a predetermined angle;
b. wrapping with a first laminar silk scaffold strip circumferentially around a vertical axis;
c. wrapping with a second laminar silk scaffold strip circumferentially around the first laminar silk scaffold strip in a manner such that the aligned porous structures of the second laminar silk scaffold strip are substantially oriented at an angle of about 20° to about 160° with respect to the aligned porous structures of the first laminar silk scaffold strip, thereby producing an implantable intervertebral disc device comprising a silk-based toroidal disc scaffold formed from the first and the second laminar silk scaffold strips.

13. The method of claim 12, wherein the angle of about 20° to about 160° includes an angle of about 80° to about 140°, or about 110° to about 130°, or an angle of about 120°.

14. The method of claim 12, wherein the laminar silk scaffold strips are produced by a method comprising:
a. Exposing a silk fibroin solution to unidirectional freezing; and
b. lyophilizing the frozen silk fibroin solution, thereby forming a laminar silk scaffold.

15. The method of claim 14, wherein the method of producing the laminar silk scaffold strips further comprises reducing the laminar silk scaffold into the first and the second laminar silk scaffold strips.

16. The method of claim 14, wherein when the silk fibroin solution further comprises a water-soluble pore-forming agent, the method of producing the laminar silk scaffold strips further comprises removing the water-soluble pore-forming agent from the laminar silk scaffold.

17. The method of claim 16, wherein the water-soluble pore-forming agent includes sodium alginate.

18. The method of claim 14, further comprising post-treatment of the laminar silk scaffold to increase insolubility of the laminar silk scaffold in an aqueous solution.

19. The method of claim 12, wherein the wrapping around the vertical axis comprises wrapping with the first laminar silk scaffold strip circumferentially around a substantially circular disc element having the vertical axis.

20. The method of claim 19, wherein the substantially circular disc element comprises a biocompatible gel.

21. The method of claim 20, wherein the biocompatible gel comprises silk fibroin.

22. The method of claim 12, further comprising seeding at least one intervertebral disc cell into the device.

23. An implantable intervertebral disc device produced by the method of claim 12.

24. A method of treating a disease or disorder associated with degeneration of an intervertebral disc in a subject comprising replacing the degenerated intervertebral disc of the subject with an implantable intervertebral disc device of claim 1.
